(19) United States
(12) Patent Application Publication
(10) Pub. No.: US 2009/0081655 A1
(43) Pub. Date: Mar. 26, 2009

(54) Multiplex Real-time Quantitative PCR

(75) Inventors: Stavros Therianos, Rochester, NY (US); Min Zhu, Rochester, NY (US); Paul Coleman, Pittsford, NY (US)

Correspondence Address: Ballard Spahr Andrews & Ingersoll, LLP, SUITE 1000, 999 PEACHTREE STREET, ATLANTA, GA, 30309-3915, US

(73) Assignee: Angela Maria Bertagna

(21) Appl. No.: 12/045,567

(22) Filed: Mar. 10, 2008

Related U.S. Application Data
(63) Continuation of application No. 10/496,626, filed on Nov. 24, 2004, now abandoned.
(60) Provisional application No. 60/336,095, filed on Nov. 30, 2001.
(60) Provisional application No. 60/397,475, filed on Jul. 19, 2002.

(51) Int. Cl. C12Q 1/68

(52) U.S. Cl. 435/6

(57) ABSTRACT

Disclosed are compositions and methods for analyzing multiple nucleic acids using PCR.

This application claims priority to U.S. Provisional Application No. 60/336,095 filed on Nov. 30, 2001, entitled “Multiplex Real-Time Quantitative PCR,” and U.S. Provisional Application No. 60/397,475 filed on Jul. 19, 2002 entitled “Multiplex Real-Time Quantitative PCR,” which applications are both herein incorporated by reference in their entirety.

### I. BACKGROUND

The development of techniques such as cDNA and large oligonucleotide array hybridization allow the transcript level analysis of thousands of genes in a single experiment. These approaches form the backbone of functional genomics. Nevertheless, the authenticity of the results obtained with such approaches has been challenged due to the limit of the techniques or the difficulty to define appropriate controls. Thus, confirming cDNA and oligoarray analysis with alternative methods is needed. There is also a need to define and validate early diagnosis of diseases, for example neurodegenerative diseases, such as Alzheimer's disease, by analyzing differential gene expression patterns. For Alzheimer's disease the only reliable diagnosis is post-mortem. An early, pre-clinical and relatively non-invasive diagnosis could improve the efficacy of currently available therapies to delay and even prevent the devastating clinical symptoms associated with such diseases. DNA amplification procedures typically are not used to quantitatively analyze clusters of genes or populations of cells because existing methods have focused on using different fluorophores, of which only four have been identified, and therefore this limits the number of genes that can be analyzed. Thus, a PCR approach that can analyze many genes, for example 50, 100, or more, in a single analysis is needed. Disclosed are Multiplex Real-Time Quantitative PCR reagents and methods that address these needs.

### II. SUMMARY

Disclosed are compositions and methods that to the analysis of more than one gene transcript in a given sample.

Additional advantages and embodiments are set forth in part in the description which follows. It is to be understood that both the foregoing general description and the following detailed description are exemplary and explanatory only and are not restrictive.

### BRIEF DESCRIPTION OF THE DRAWINGS

FIG. 1 shows a general scheme for performing the disclosed methods, an overview of Single Channel Quantitative Multiplex RT-PCR (ScqmRT-PCR). It is typically composed of 6 possible steps. Steps 1-5 typically (Primer design and subcloning; standard curve construction; RNA extraction and reverse transcription; first round PCR; and second round real time quantitative PCR) are typically performed and step 6 typically (which consists, for example, of principal component analysis; canonical analysis; array comparison) can be adapted depending on what type of analysis is needed.

FIG. 2 shows validation steps of scqmRT-PCR. FIG. 2A, 19 targets were processed in parallel from the same amount of starting material and a representative example of threshold cycles obtained during the quantitative round of PCR is shown. FIG. 2B, standard curves related to the 19 target transcripts were constructed derived from the threshold cycles. FIG. 2C, 1% agarose gel run after the quantitation as a demonstration of amplicons specificity. Such gels typically are run after each experiment. FIG. 2D, comparison of “regular” quantitative RT-PCR and scqmRT-PCR. The upper trend line was obtained following a regular quantitative RT-PCR protocol where 10⁴ copies of starting material were considered as an unknown copy number on the thermocycler settings. The lower standard curve represents the same experiment performed using scqmRT-PCR protocol. The same starting mRNA copy number was obtained using both techniques. FIG. 2E-F shows two representative examples of standard curves performed during the second round of PCR. FIG. 2E represents a homeobox HOXB7 transcript and FIG. 2F represents a standard curve for the octamer-binding transcript Oct-3. Note the sensitivity obtained for HOXB7 (10 plasmid copies) and the correlation coefficient obtained for Oct-3 (0.999).

FIG. 3 shows mRNA copy number per fig total RNA comparisons from control, intermediary and AD cases. FIG. 3A, 7 transcripts showed consistent change between controls (in black) and AD cases (in mid-gray). Note that the intermediary cases (in light gray) matched closer to the AD group. AP180, Dynamin, Syntaxin, ICAM5 and CamK2G are related to the dendritic and the synaptic apparatus. EGR1 showed a greater heterogeneity within the Control group. FIG. 3B, 8 transcripts displayed heterogeneity in their mRNA copy numbers in the 3 groups. FIG. 3C, 3 transcripts that showed higher homogeneity within control and intermediary cases compared to AD cases. FIG. 3D shows a representative agarose gel performed after the second round of PCR (the actual quantitative round). Each lane represents a different candidate (for example lane 1 corresponds to beta-actin). The PCR products are virtually devoid of any primer dimers and there is no unspecific amplification.

FIG. 4 shows principal component analysis performed on scqmRT-PCR results. FIG. 4A, 2 dimensional plot constructed based on the entire set of genes (Mao, Y., et al., (2001), Cell 104, 433-440). Cases clustered according to their disease status and intermediary cases (in light gray) were positioned closer to the AD cases (in mid-gray) than to the control cases (in black). FIG. 4B, Same analysis achieved with AP180, PP2CB, Dynamin, Syntaxin, ICAM5, PARG and CamK2G. This set of transcripts was sufficient to separate control cases from AD cases and the intermediary cases clustered closer to the AD group. FIG. 4C, Relative importance of principal components for the 19 candidate genes. Note that the first 2 components accounted for 75.5% of the variance among the cases. FIG. 4D, Relative importance of principal components for 7 candidate genes including AP180, PP2CB, Dynamin, Syntaxin, ICAM5, PARG and CamK2G. Here the first 2 components accounted for 92.1% of the variance among the cases.

FIG. 5 shows a comparison of micro-arrays and scqmRT-PCR. FIG. 5A, Fold changes between control and AD cases measured with either micro-array data or scqmRT-PCR showed inconsistencies for several candidates including FKHR, Integrin 5, Oct 3 and PECAM 1. FIG. 5B, scqmRT-PCR 2 dimensional plot of principal components constructed with 18 genes that are also present of micro-arrays. Note that the intermediary cases (in light gray) clustered with AD cases (in mid-gray). FIG. 5C, same analysis as for B but based on micro-arrays indirect fluorescence index. Here, the 2 intermediary cases were not discernible from controls despite their AD histological pathology.

FIG. 6 shows information related to an exemplary set of primers which could be used to analyze transcript information in cells with abberent proliferation, such as cancer cells.

FIG. 7 shows information related to the primer sequences for the second PCR used to analyze transcripts for Alzheimer's Disease and other neurological disorders discussed in Example 1.

FIG. 8 shows information related to the primer sequences for the first PCR used to analyze transcripts for Alzheimer's Disease and other neurological disorders discussed in Example 1.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. COMPOSITIONS AND METHODS

Effective approaches using array technologies are critical to understand the molecular bases of human diseases. In the context of Alzheimer's Disease, where the identification of molecular mechanisms of underlying pathologies is vital, disclosed is an assay which is a real time RT-PCR based high throughput approach that can simultaneously quantify the expression of a large number of genes at the copy number level from a minute amount of starting material. Using this approach within the human brain, 19 genes at a time were quantified with only one type of fluorescent probe. The number of genes included can be considerably increased. Examples of consistent changes in AD within these 19 candidate genes included reductions in targets related to the dendritic and synaptic apparatus. Also disclosed is comparison data with microarray analysis from the same brain region and the same subjects. These techniques can be widely used for diagnostic purposes as well as basic research.

Simultaneous quantitation of numerous transcripts extracted from a defined tissue sample provides fundamental information for molecular neurobiology. Within identified states of a disease, such information helps the understanding of molecular cascades underlying pathologies. Disclosed are methods that would allow the coincident expression profiling and analysis of a large number of genes at the copy number level and from minute quantities of starting material. Disclosed is a single channel quantitative multiplex RT-PCR (scqmRT-PCR). Disclosed are methods that can be performed using only one fluorescent reporter probe which helps in avoiding the high background encountered in traditional multi-channel multiplex quantitative PCR methods. The uniformity, sensitivity, and specificity of the disclosed methods is equivalent to that of single transcript real-time PCR (Freeman et al., 1999).

The disclosed methods and compositions are designed to allow simultaneous analysis of the expression of a number of different genes. The disclosed methods are capable of quantifying the relative and absolute amounts of the targeted genes. Current available methods only provide semi-quantitative or qualitative gene expression level by using fluorescence intensity as an indirect index, such as in microarrays, or the methods are limited to the analysis of typically less than 5 genes at a time because they are restricted by the number of different fluorescence channels widely available (Bustin, S. A., (2000) J Mol Endocrinol 25, 169-193.). The disclosed methods, while they can be used with more than one reporter, can function with only one reporter signal. When the disclosed methods are used with more than one reporter the number of genes which can be analyzed increases accordingly. The requirement of only one fluorescent reporter avoids the high background encountered in other systems for looking at more than one gene at a time.

Disclosed herein is a PCR-based high-throughput method for simultaneously analyzing the expression of multiple genes. The method can use minute quantities of starting material and reach single copy levels of efficiency, for example, where only a single target nucleic acid was available, such as a single copy of transcript from a single target cell. For example, for the analysis of 20 transcripts in triplicate for 4 subjects, less than 1 μg total RNA per subject is needed. The disclosed methods are capable of simultaneously analyzing multiple genes. The disclosed methods use gene-specific primers in particular ways. The disclosed methods can quantify multiple genes with the use of a single signal reagent, such as a fluorescent probe.

In general, the method is useful for obtaining quantitative information about the expression of many different genes in a sample that can contain as little as a single cell. Since the disclosed methods are quantitative, comparisons of the expression patterns at a quantitative level between a variety of different cell states or cell types can be achieved. In general, total RNA can be isolated from the target sample using any isolation procedure. This RNA can then be used to generate first strand copy DNA (cDNA) using any procedure, for example using random primers or oligo-dt primers or random-oligo-dt primers which are oligo-dt primers coupled, on the 3′ end, to short stretches of specific sequence covering all possible combinations, so the primer primes at the junction between the polyA tract and non-poly A tract associated with messenger RNA (mRNA). The cDNA is then used as a template in a PCR reaction. This PCR reaction is performed with primer pairs, a forward and a reverse primer, that are specific for the expressed genes, which are to be tracked. This reaction can contain as many different primer pairs as desired, but typically would include between 5 and 100 different sets of primers, each specific for a single gene or single isoform (including any specific number between 5 and 100). Typically all of the primers will be in about equimolar concentration. After performing a number of PCR cycles, for example 15 cycles, such that the DNA is still amplifying at about greater than 80% or 85% or 90% or 95% the doubling rate, the PCR is stopped. Typically, in the first round of PCR, if quantitative PCR (real time PCR) was performed, you do not reach the threshold cycle of amplification. However, the disclosed methods in certain embodiments can still work if amplification proceeds for about less than 9 or 8 or 7 or 6 or 5 or 4 or 3 or 2 or 1 cycle(s) past the threshold cycle. The number of cycles in the first round depends on the amount of starting materials. For example, 20 cycles can be used for single cell experiments. The PCR reaction is then partitioned into new reaction tubes for a (new) second round of PCR. Each of the tubes contains a fraction of the previous PCR reaction mixture which contains all of the products produced from all of the specific primers present in the first PCR mixture. In the second PCR mixture, containing the fraction of the first PCR mixture, typically only one of the specific primer pairs or a new primer pair is added, in addition to the universal primer which has the molecular beacon attached, and the PCR is performed. Typically this second round of PCR is performed using quantitative real time PCR protocols, which for example, rely on increases in fluorescence at each cycle of PCR through, (for example, probes that hybridize to a portion of one of the amplification probes) the release of fluorescene from a quencher sequence while the uniprimer (universal primer) binds to the DNA sequence. Fluorescence approaches used in real-time quantitative PCR are typically based on a fluorescent reporter dye such as SYBR green, FAM, fluorescein, HEX, TET, TAMRA, etc. and a quencher such as DABSYL, Black Hole, etc. When the quencher is separated from the probe during the extension phase of PCR, the fluorescence of the reporter can be measured. Systems like Molecular Beacons, Taqman Probes, Scorpion Primers or Sunrise Primers and others use this approach to perform real-time quantitative PCR. Examples of methods and reagents related to real time probes can be found in U.S. Pat. Nos. 5,925,517; 6,103,476; 6,150,097, and 6,037,130, which are incorporated by reference herein at least for material related to detection methods for nucleic acids and PCR methods. In addition to performing the above steps, the generation of a standard curve for the primer pairs, and typically for each individual primer pair, should be made so that data obtained from the second round of PCR can be accurately correlated with an absolute copy number of the original starting material in the target sample, containing for example, the target cell or cells. Each of these steps of the general method, as well as the reagents and variations of the method, are discussed in detail herein. A key aspect to understanding the disclosed methods is the combination of a first PCR containing the multiple different primer pairs in a batch PCR mixture in which all target gene products or fragments of gene products are amplified with a second PCR panel in which the specific amplification reaction occurs in which a portion of the batch PCR mixture is amplified with specific primer pairs. Quantitation is typically achieved by reference to a standard curve that is generated for the complete primer pairs or each individual primer pair.

Disclosed are methods that use 1 μg of total RNA which allows the number of transcripts to be analyzed in parallel to be increased based on the following facts: first only 5% of the reverse transcribed material was used to perform the analysis; second for each second round of PCR reaction only 1% of the first round products was used. Therefore, at least, up to 500 targets, or more, could be analyzed in parallel from 1 μg of total RNA. Alternatively, the quantity of starting material could be highly reduced to the level of single cell transcript amplification. Disclosed are methods that can be used in conjunction with other single cell technologies such as aRNA amplification (Miyashiro et al., 1994). The combination of different techniques with the disclosed methods can increase the precision of single cell techniques.

The disclosed methods can be used with any type of detection system. For example, “sunrise” primers that contain a universal sequence on their 5′ end (Nazarenko et al., 1997) can be used as well as a ‘molecular beacon’ approach (Taqman) without great modifications (Bustin, 2000). There are numerous ways to identify nucleic acids and all of these ways can work with the disclosed methods, within the boundaries of each technique.

Also, standard curves can be used in the disclosed methods, but other methods to derive absolute copy number of targets, such as analysis using C(t) can also be used.

Comparisons of gene expression between normal aging and neurodegenerative diseases is frequently hampered by the fact that “housekeeping” genes such as GAPDH and β-actin, often used as reference values, are changed during the course of the disease. Altered expression of these genes in AD is consistent with the metabolic and structural changes known to occur in AD (Braak et al., 1999; Dickson, 2001; Perl, 2000). The problems induced by the use of “housekeeping” genes as reference standards are reduced by the quantification of absolute copy number produced by the disclosed methods. Thus, by bringing together quantitation at the copy number level for each target and quantitation of numerous targets in parallel, disclosed is a set of genes, largely related to the dendrite and the synapse, that yield data that are consistently changed in AD. This is in accordance with findings that the dendritic and synaptic machinery is undoubtedly affected in AD (Small et al., 2001).

The disclosed methods can be used to analyze the expression pattern of a group of genes to separate different disease subtypes has been a promising approach for clinical diagnosis (Dhanasekaran et al., 2001; Pomeroy et al., 2002; van't Veer et al., 2002). However, current publications were derived mainly from microarray studies, which are restricted from practical application for a number of reasons (time, costs, etc.). The disclosed methods provide more flexibility in choosing candidate genes and allow robust separation of groups with a significantly smaller number of genes. Indeed the coupling of scqmRT-PCR with multivariate statistical analyses such as PCA, can be used for the early identification of any disease, not limited to neurodegenerative disorders. Based on the disclosed results with 7 transcripts related to the dendritic and synaptic apparatus, the disclosed combination of molecular and statistical tools, displayed by the use of scqmRT-PCR coupled with PCA, can be used to discriminate between age-matched control and intermediary AD cases. As an example, disclosed herein intermediary cases that did not meet clinical criteria for AD but did meet neuropathological criteria for AD at autopsy were separated from controls based on their gene expression. In fact, this kind of test could be a prerequisite for any large-scale analysis in the sense that it could rapidly separate different populations of interest at a molecular level.

AD is a complex, dichotomous and heterogeneous disease (Tanzi and Bertram, 2001). Both based on a pathobiological and a genetic linkage approach, the search for “strong AD candidates” now relies heavily on the use of large-scale microarrays. It is also widely recognized that although clearly essential, array approaches need independent confirmation that will distinguish among consistent, inconsistent or likely false positive/negative findings. scqmRT-PCR complies with the parameters of such an independent experimental technique that will allow validation of microarrays. Disclosed herein, the evaluation of transcripts predicted to be enriched or diminished in AD based on microarrays data was confirmed only for roughly half of the candidates. It should be pointed out that most of the candidates showed a consistent change on arrays despite the fact that the fold changes were not important. In other words, these changes were robust and reproducible within the samples studied using Affymetrix technology but not confirmed when tested with scqmRT-PCR. When these transcripts were analyzed as a whole using PCA, both technologies were able to separate controls from AD cases but arrays lacked the level of precision necessary to distinguish cases that were “transitional”. In other words, array analysis gave rise to an accurate description and scqmRT-PCR added to this a level of prediction in the test that was absent from the previous approach. These predictions, being in this case, corroborated by post-mortem diagnosis.

Thus, disclosed are methods that allow for increased precision in making a molecular diagnosis of disease, such as Alzheimer's disease. The disclosed methods allow for the quantitation of many different genes.

1. Methods

a) General Method

The general method is drawn to quantitative analysis of the gene expression patterns of multiple genes in a single analytical event. The need for this type of method is great. Existing methods only rely on qualitative analysis because of the inability to accurately track multiple genes at a single time using amplification methods. Furthermore, semi quantitative means that rely on hybridization, for example, chip technology and micro arrays, need ways to validate the multiplexing abilities. Thus, the disclosed methods provide a quantitative means that relies on nucleic acid amplification techniques.

In general the method can employ a reverse transcription step to produce cDNA, a first PCR reaction step performed with multiple different specific primer pairs which are specific for different target gene expression transcripts, wherein the first PCR generates all of the target products at the same time, a second PCR step performed with only one of the specific primer pairs on an aliquot of the first PCR mixture which is typically performed in parallel with second PCRs of all of the other individual specific primer pairs, and a step of comparing the PCR product amounts obtained from the second PCR to a standard curve generated for the specific primer pair or a representative standard curve generated from the unique primer pair.

Disclosed are methods of quantifying a target nucleic acid in a sample, comprising 1) performing a first PCR comprising a first set of PCR primer pairs that produces a set of first PCR products, 2) performing a second PCR comprising a second primer pair and an aliquat of the first set of PCR products that produces a second PCR product, 3) producing a standard curve for each PCR product produced from an aliquat of the first set of PCR products, and 4) comparing the second PCR product to the standard curve.

in a PCR mixture, wherein the mixture comprises a group of target nucleic acid molecules and a group of first PCR primer pairs, wherein each primer pair is designed to amplify a region of one of the target nucleic acid molecules in the group of target nucleic acid molecules, wherein the PCR produces a first group of PCR products related to the target nucleic acid molecules, 2) performing a second PCR in a PCR mixture, wherein the mixture comprises an aliquot of the first group of PCR products and a single primer pair which is designed to amplify one of the target nucleic acid products, wherein the second PCR produces a second target nucleic acid PCR product related to one of the target nucleic acid molecules, and 3) quantifying the number of copies of the second target nucleic acid product present in the sample containing the target nucleic acid molecule.

Disclosed are methods of determining the relative number of copies of a group of target nucleic acid molecules present in a sample containing the target nucleic acid molecules, comprising 1) performing a first PCR in a PCR mixture, wherein the mixture comprises a group of target nucleic acid molecules and a group of first PCR primer pairs, wherein each primer pair is designed to amplify a region of one of the target nucleic acid molecules in the group of target nucleic acid molecules, wherein the PCR produces a first group of PCR products related to the target nucleic acid molecules, 2) performing a second PCR in a PCR mixture, wherein the mixture comprises an aliquot of the first group of PCR products and a single primer pair which is designed to amplify one of the target nucleic acid products, wherein the second PCR produces a second target nucleic acid PCR product related to one of the target nucleic acid molecules, and 3) quantifying the number of copies of the second target nucleic acid product present in the sample containing the target nucleic acid molecule.

Also disclosed are methods, wherein each first PCR primer pair comprises one forward primer and one reverse primer, wherein the forward and reverse primers are about equimolar, wherein each first primer PCR set is about equimolar to each of the other first PCR primer pairs in the group of first PCR primer pair, and/or wherein each first PCR primer has about a 50% GC content.

Also disclosed are methods, wherein the first PCR is started by a hot-start.

Also disclosed are methods, wherein a first target nucleic acid product is less than 500 nucleotides long or wherein each first target nucleic acid product is less than 500 nucleotides long.

Also disclosed are methods, wherein the first PCR is performed with at least two sets of gene specific primers or wherein the second PCR is performed with one set of gene specific primers.

Also disclosed are methods, wherein in the first PCR the primer pairs are equimolar or wherein each primer pair in the group of primer pairs are equimolar to each other.

Also disclosed are methods wherein the primer pair in the second PCR is different than the any of the primer pairs in the first PCR, wherein the primer pairs in the second PCR contain a universal primer sequence.

Also disclosed are methods, wherein the first group of PCR products was derived from at least 5 different target nucleic acid molecules.

Also disclosed are methods, wherein the products produced from the target nucleic acid molecules are all between 177 and 237 nucleotides long but could range up from 20 to 1500 base pairs

Also disclosed are methods, wherein the single primer pair is a primer pair not present in the group of first PCR primer pairs, wherein the single primer pair is a primer pair present in the group of first PCR primer pairs, or wherein the second PCR has at least one single primer pair which is different and at least one single primer pair which is the same as a first primer pair.

Also disclosed are methods, wherein one of the primers from the single primer pair in the second PCR, interacts with or is derived from a fluorescent reporter.

Also disclosed are methods, wherein the fluorescent reporter is selected from the group consisting of SYBR green, Taqman probe, Molecular Beacon, Scorpion Primer, Sunrise Primer and Eclispe Probe.

Also disclosed are methods, wherein the fluorescence reporter probe is coupled with a quencher.

Also disclosed are methods, wherein quantifying the number of copies of the target nucleic acid molecule related to the second PCR product present in the sample containing the target nucleic acid molecule comprises comparing the amount of the second PCR product to a standard curve.

Also disclosed are methods, wherein the standard curve is specific for the second PCR product.

Also disclosed are methods, further comprising producing cDNA related to the target nucleic acid molecules before performing the first PCR.

Also disclosed are methods, further comprising producing RNA prior to producing the cDNA.

### (1) Preparation of the RNA

The disclosed methods typically involve some level of RNA preparation. The RNA preparation step is not required to be performed as part of a contiguous method, but the method requires a template for a PCR reaction. As the template for a PCR reaction is typically DNA and typically the target material to be analyzed is expressed mRNA, typically the starting template material for the first PCR reaction will be cDNA which was generated from purified RNA including mRNA. While in theory, the RNA preparation step could be performed far removed from the actual amplification and quantitation steps, for example, in another laboratory, or at a much earlier time, in many embodiments the RNA isolation and preparation of the cDNA will occur in conjunction with the amplification and quantitation steps of the methods, but this is not required. It is understood, however, that the method can be performed on existing cDNA libraries, for example, and other existing DNA libraries.

When an RNA preparation step is included in the disclosed methods, the method of RNA preparation can be any method of RNA preparation that produces enzymatically manipulatable mRNA. For example, the RNA can be isolated by using the guanidinium isothiocyanate-ultracentrifugation method, the guanidinium and phenol-chloroform method, the lithium chloride-SDS-urea method or poly A+/mRNA from tissue lysates using oligo(dT) cellulose method (See for example, Schildkraut, C. L., et al., (1962) J. Mol. Biol. 4, 430-433; Chomczynski, P., and Sacchi, N. Anal. Biochem. 162, 156 (1987); Auffray and F. Rougeon (1980), Eur J Biochem 107:303-314; Aviv H, Leder P. (1972), Proc Natl Acad Sci USA 69, 1408-1412; Sambrook J, et al., (1989). Selection of poly A+ RNA. In: Molecular Cloning, vol. 1, 7.26-7.29. All of which are herein incorporated by reference at least for material related to RNA purification and isolation)

It is important when isolating the RNA that enough RNA is isolated. Furthermore, typically the quantity of RNA obtained can be determined. For example, typically at least 0.01 ng or 0.5 ng or 1 ng or 10 ng or 100 ng or 1,000 ng or 10,000 ng or 100,000 of RNA can be isolated. As will be discussed herein, during the amplification PCR it is important that when the amplification is stopped that the amplification of each target product that remains be at least about 80% or 85% or 90% or 95% the doubling rate. The number of cycles of PCR that are performed so as to continue to remain at about the doubling rate is related to the amount of total RNA that was used in the cDNA generation step.

The RNA can be isolated from any desired cell or cell type and from any organism, including mammals, such as mouse, rat, rabbit, dog, cat, monkey, and human, as well as other non-mammalian animals, such as fish or amphibians, as well as plants and even prokaryotes, such as bacteria. Thus, the DNA used in the method can also be from any organism, such as that disclosed for RNA.

### (2) Generation of the cDNA

The disclosed methods typically involve some level of cDNA preparation. The cDNA preparation step is not required to be performed as part of a contiguous method, but the method requires a template for a PCR reaction. As the template for a PCR reaction is typically DNA and typically the target material to be analyzed is expressed mRNA, typically the starting template material for the first PCR reaction will be cDNA which was generated from purified RNA including mRNA. While in theory, the cDNA preparation step could be performed far removed from the actual amplification and quantitation steps, for example, in another laboratory, or at a much earlier time, in many embodiments the preparation of the cDNA will occur in conjunction with the amplification and quantitation steps of the methods, but this is not required.

When a cDNA preparation step is included in the disclosed methods, the method of cDNA preparation can be any method of cDNA preparation that produces enzymatically manipulatable cDNA. For example, the cDNA can be prepared by using, for example, random primers, poly-d(T) oligos, or NVd(T) oligos. For the purpose of data normalization, an equal amount of total RNA is typically used for cDNA synthesis. Many examples exist of performing reverse transcription to produce cDNA for use in PCR, including the following: Glisin V., R. Crkvenjakov and C. Byus (1974) Ribonucleic acid isolated by caesium chloride centrifugation Biochemistry 13:2633-7; Ullrich A., J. Shine, J. Chirgwin, R. Pictet, E. Tischer, W. J. Rutter and H. M. Goodman (1977) Rat insulin genes:construction of plasmids containing the coding sequences Science 196:1313; Chirgwin J. M., A. E. Przybyla, R. J. MacDonald and W. J. Rutter (1979) Isolation of biologically active ribonucleic acid from sources enriched in ribonuclease, Biochemistry 18:5294-9; Faulkner-Jones B. E., D. S. Cram, J. Kun and L. C. Harrison (1993) Localization and quantitation of expression of two glutamate decarboxylase genes in pancreatic b-cells and other peripheral tissues of mouse and rat Endocrinol 133:2962-2972; and Gonda T. J., D. K. Sheiness and J. M. Bishop (1982) Transcripts from the cellular homologs of retroviral oncogenes:distribution among chicken tissues Mol Cell Biol 2:617-624, which are herein incorporated by reference at least for material related to DNA amplification.

### (3) First PCR

The disclosed methods include a step of performing a first PCR. The first PCR typically will be performed on molecules that potentially contain the target nucleic acid molecules. Thus, for example, the first PCR should contain target nucleic acid molecules or copies of the target nucleic acid molecules that are manipulatable by PCR, for example, DNA, for example a cDNA template, such as a commercial cDNA library or a cDNA library generated de novo for use in the disclosed method. The disclosed method typically requires that a group of primer pairs be used simultaneously during the first PCR reaction. A primer pair contains at least a forward and a reverse primer for a specific target template. A group of primer pairs includes at least two different primer pairs. A group of primer pairs can typically contain at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 160, 170, 180, 190, 200, or more primer pairs. The group of primer pairs can also contain less than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 160, 170, 180, 190, 200, or more primer pairs.

The primer pairs are specific for different target genes. A primer pair is specific if in an assay to identify the specificity of the primer run under conditions under which the primer would experimentally be used only a band corresponding to the intended product is visible on an agarose gel after an appropriate number of cycles, for example 10, 15, 20, 25, 30, 35, 40, 45, 50.

It is important that the group of primer pairs is compatible. This means that the primer pairs that make up a given group of primer pairs should not interact with each other or with a target gene other than their cognate gene. The compatibility of primer pairs can be determined using any method available to the skilled artisan. For example, there are computer programs that will use algorithms to predict whether a given set of nucleic acid sequences will interact with each other (such as DNA Strider™). Another way to determine whether the primer pairs to be used in a group are compatible is to empirically test the primer pairs against each other and modify as needed. For example, doing qualitative multiplex PCR with all the primers designed and running the PCR products on an agarose gel can yield information. Primer pairs are considered compatible if only bands corresponding to each PCR product are produced. One can determine whether the primer pairs to be used in a group are compatible by empirically testing the primer pairs against each other and modifying as needed. The primer pairs typically will have about the same melting temperature. The primer pairs also will typically have about 50% GC content. It is also typical that each primer pair within a group of primer pairs will have about the same melting temperature to each other primer pair. Likewise, it is typical for each primer pair within a group of primer pairs to have about 50% GC content. The length of the primers is typically between about 10 and about 30 nucleotides, but can be any length that functions to amplify the DNA. The primers are typically less than about 60, 55, 50, 45, 40, 35, 30, 25, 20, 15, or 10 nucleotides long.

The first PCR produces a product, which is typically a region of the target gene transcript of interest rather than the full length of the gene. For example, typically the product produced from the first PCR will be less than about 1500, 1400, 1300, 1200, 1100, 1000, 900, 800, 700, 600, 500, 400, 300, 250, 225, 200, 175 or 150 nucleotides long.

The product can also range between for example, about 100 and about 2000 nucleotides long or about 200 and about 1500 nucleotides long or about 100 and about 300 nucleotides, for example. All other possible permutations where the number of nucleotides of the longest member is up to about 3000 nucleotides long are also disclosed herein. As an example, typically to be compatible with the molecular beacon system, the PCR product should be between 180-250 bp nucleotide long.

In certain embodiments the first PCR is started by a hot start. There are many ways to perform a hot start, but in general, a hot start simply means that before the first time extension of the primers is performed, the PCR mixture is heated for a period of time at a high temperature, such as 95 degrees C. The period of time can vary, but in general the time will be long enough to destroy any residual non-thermal stable polymerases which may be present in the mixture, for example, at least 5 minutes or 10 minutes or 15 minutes at 95 degrees C.

The first PCR can be performed using any conditions appropriate for the primer pairs and templates being used. For example, the concentration of the dNTPs or primers or enzyme or buffer conditions can be any concentration that allows the PCR to occur. Typically the concentration of the dNTPs can be between 2.5 and 10 mM each. Typically the concentration of the primers can be between 0.1 and 0.5 μM each, however, typically the primer pairs will be at about the same concentration, i.e. equimolar. Typically the concentration of the enzyme can be between 1 to 3 units per reaction. Typically the concentration and make up of the buffers is for example 1× final concentration out of a 10× stock solution as suggested by the manufacturer of the thermal polymerase. But it is understood that conditions other than these can also work, and in some cases may be determined after empirical testing.

Any type of thermal stable polymerase can be used. If a hot-start is going to be performed it is preferred that the thermal stable polymerase be of the type that is not functional until after an extended period of incubation at a high temperature, such as greater than 90 degrees C.

The number of cycles that is performed in the first PCR is related to the amount of starting material present at the start of the first PCR. As discussed herein each primer pair and product produced from the primer pair has a certain doubling rate that is related to the conditions that the amplification is occurring in. In any given PCR there is a doubling rate associated with the production of the product, and as cycles of PCR continue, there comes a point at which the amount of DNA produced at each cycle of PCR begins to decrease below the doubling rate for that reaction. This phenomena is related to, for example, the loss of free primer in the reaction mixture. At the first cycle of PCR there is a significant excess of primer over template, but as the template is amplified and each cycle of PCR uses more of the remaining primer, there comes a point where the primer hybridizing to free template becomes limiting, and this can decrease the amount of amplification that takes place during that cycle of the PCR.

The disclosed method typically will be performed so that the first PCR is stopped, i.e., no more cycles of PCR are performed, before a significant decrease in the amplification rate occurs. Thus, typically the first PCR is stopped before the amplification is less than or equal to about 97%, 95%, 90%, 85%, 80%, 75%, 70%, or 65% of the doubling rate. In certain embodiments, the PCR is stopped when the amplification rate is greater than or equal to about 97%, 95%, 90%, 85%, 80%, 75%, 70%, or 65% of the doubling rate.

It is understood that the doubling rate for a given sample can be determined empirically, but doubling rates of 1.99, 1.98, 1.97, 1.96, 1.95, 1.94, 1.93, 1.92, 1.91, 1.90, 1.89, 1.88, 1.87, 1.86, 1.85, 1.84, 1.83, 1.82, 1.81, 1.80, 1.79, 1.78, 1.77, 1.76, 1.75, 1.74, 1.73, 1.72, 1.71, 1.70, 1.65, 1.60, 1.55, 1.50, 1.40, and 1.30 can be seen.

It is understood that doubling rates about these numbers as well as greater than or less than or greater than or equal or less than or equal are also disclosed.

Typically the number of cycles of the first PCR is related to the amount of starting material that is used, and in certain situations, the starting material is cDNA produced from total RNA which was prepared from a sample of cells. The starting material could also be a mixture of starting DNA. It can be empirically determined, for example, that about 95% of the doubling rate remains after 15 cycles of PCR when the amount of RNA isolated and used to produce the first strand cDNA is about 1 μg. Typically 10-15 cycles will retain greater than 95% of the doubling rate when using 100 ng to 3 μg of total RNA. Typically the starting quantities of total RNA will be less than or equal to about 20 μg or 15 μg or 10 or μg or 9 μg or 8 μg or 7 μg or 6 μg or 5 μg or 4 μg or 3 μg or 2 μg or 1 μg or quantities of RNA that can be present in a single cell. The disclosed methods can be used with less RNA than other methods, such as a Northern Blot analysis, which typically will need at least 10 μg of total RNA to produce data for a single transcript. The disclosed method can use as little as single copy numbers of transcripts contained within a total RNA sample. The only limiting factor for the lower limit of RNA amounts is as the total amount of RNA isolated is decreased the probability of losing any given transcript that is present in low copy numbers increases. For example, as the amount of RNA used decreases, eventually an amount would be reached that because of probabilities would not contain a single copy of a transcript that was originally in low copy number. While there is a not an absolute amount of RNA where this will occur in all situations, an amount of RNA greater than about 30 ng or 35 ng or 40 ng or 45 ng or 50 ng or 55 ng or 60 ng or 65 ng or 70 ng or 75 ng or 80 ng typically will not encounter problems of loss of low copy number transcripts. However, when less RNA then this is used, repetitions of the analysis can adjust for the potential loss of single copy transcripts.

It is also reasonable, while not required to optimize the conditions of the first PCR. This would typically entail, a series of PCRs performed under different conditions, typically done in parallel to identify the best temperatures, times, primer concentrations, and number of PCR cycles, for example, that should be used.

After the first PCR has been completed, there is a mixture of products present in the PCR that relates to the starting target nucleic acid molecules as determined by the target specific primer pairs used. As the amount of DNA at the end of a PCR is determined in part by the amount of starting template present in the mixture, and as the amount of starting material for the target nucleic acids will typically be different, the amount of product material for each target nucleic acid will typically be different. Quantitation does not typically occur at this point in the disclosed methods. Qualitative assessment of the differences in the amount of product can be obtained by, for example, analyzing the products with polyacrylamide gel electrophoresis or regular PCR and agarose gel. Examples exist for performing qualitative multiplex PCR, a few of which are set forth in the following references: Audinat E, Lambolez B, Rossier J, Crepel F (1994) Activity-dependent regulation of N-methyl-D-aspartate receptor subunit expression in rat cerebellar granule cells. Eur J Neurosci 6:1792-1800; Audinat E, Lambolez B, Rossier J (1996) Functional and molecular analysis of glutamate-gated channels by patch-clamp and RT-PCR at the single cell level. Neurochem Int 28:119-136; Audinat E, Lambolez B, Cauli B, Ropert N, Perrais D, Hestrin S, Rossier J (1996) Diversity of glutamate receptors in neocortical neurons: implications for synaptic plasticity. J Physiol Paris 90:331-332; Bochet P, Audinat E, Lambolez B, Crepel F, Rossier J (1993) Analysis of AMPA receptor subunits expressed by single Purkinje cells using RNA polymerase chain reaction. Biochem Soc Trans 21:93-97; Bochet P, Audinat E, Lambolez B, Crepel F, Rossier J, Iino M, Tsuzuki K, Ozawa S (1994) Subunit composition at the single-cell level explains functional properties of a glutamate-gated channel. Neuron 12:383-388; Cauli B, Porter J T, Tsuzuki K, Lambolez B, Rossier J, Quenet B, Audinat E (2000) Classification of fusiform neocortical interneurons based on unsupervised clustering. Proc Natl Acad Sci U S A 97:6144-6149; Crepel F, Audinat E, Daniel H, Hemart N, Jaillard D, Rossier J, Lambolez B (1994) Cellular locus of the nitric oxide-synthase involved in cerebellar long-term depression induced by high external potassium concentration. Neuropharmacology 33:1399-1405; Curutchet P, Bochet P, Prado de Carvalho L, Lambolez B, Stimnakre J, Rossier J (1992) In the GluR1 glutamate receptor subunit a glutamine to histidine point mutation suppresses inward rectification but not calcium permeability. Biochem Biophys Res Commun 182:1089-1093; Johansen F F, Lambolez B, Audinat E, Bochet P, Rossier J (1995) Single cell RT-PCR proceeds without the risk of genomic DNA amplification. Neurochem Int 26:239-243; Lambolez B, Audinat E, Bochet P, Crepel F, Rossier J (1992) AMPA receptor subunits expressed by single Purkinje cells. Neuron 9:247-258; Lambolez B, Ropert N, Perrais D, Rossier J, Hestrin S (1996) Correlation between kinetics and RNA splicing of alpha-amino-3-hydroxy-5-methylisoxazole-4-propionic acid receptors in neocortical neurons. Proc Natl Acad Sci USA 93:1797-1802; Potier M C, Dutriaux A, Lambolez B, Bochet P, Rossier J (1993) Assignment of the human glutamate receptor gene GLUR5 to 21q22 by screening a chromosome 21 YAC library. Genomics 15:696-697; Ruano D, Lambolez B, Rossier J, Paternain A V, Lerma J (1995) Kainate receptor subunits expressed in single cultured hippocampal neurons: molecular and functional variants by RNA editing. Neuron 14:1009-1017; and Tsuzuki K, Lambolez B, Rossier J, Ozawa S (2001) Absolute quantification of AMPA receptor subunit mRNAs in single hippocampal neurons. J Neurochem 77:1650-1659, which are incorporated herein by reference at least for material related to methods related to nucleic acid amplification. The precision of quantitation provided by the present method occurs with the coupling of this first PCR to the second PCR and the comparison of the product in the second PCR to a standard curve for the reaction mixture or primer pairs.

### (4) Second PCR

In general, typically the number of second PCR mixtures that are utilized in the second PCR will be equal to the number of different first PCR primer pairs within the group of first PCR primer pairs. While the first PCR had a group of primer pairs in one reaction mixture, the second PCR typically has a single or reduced number of primer pairs present in a single reaction mixture, and typically there will be multiple second PCR reaction mixtures, each with a different group of primer pairs or different individual primer pair. For example, if in the first PCR there were 30 different first PCR primer pairs in the group of first PCR primer pairs, then typically there would be 30 separate second PCRs that are each designed to amplify the major product produced from one of the first PCR primer pairs. In another example, if in the first PCR there were 50 different first PCR primer pairs in the group of first PCR primer pairs, then typically there would be 50 separate second PCRs that are each designed to amplify the major product produced from one of the first PCR primer pairs. It is not required that every specific first PCR product ultimately be amplified in a second PCR as discussed herein. It is understood that more than one second PCR primer pair can be present in the second PCR and quantified if there is a way to quantify each product produced in the second PCR. For example, 4 separate second PCR primer pairs could be used in the second PCR if 4 separate fluorophores are used. However, the ability to quantify the number of copies of the target nucleic acid molecule related to a particular first PCR product typically occurs through the second PCR and subsequent analysis. To add certainty and to test reproducibility, triplicates for each second PCR can be performed.

The disclosed methods thus, typically comprise a second PCR. The second PCR is related to the first PCR in that the starting template for the second PCR comes at least from the PCR product produced in the first PCR reaction. Typically this will be accomplished by taking an aliquot from the first PCR mixture after the first PCR mixture has undergone at least one cycle of PCR. This aliquot typically will be a fraction of the first PCR mixture that has undergone at least one cycle of PCR, such as 1/100, or 1/50, or 1/25, or 1/10 of the first PCR mixture. In general, the aliquot of the first PCR mix will be less than or equal to (1/(the number of primer pairs)). Typically the aliquots from the first PCR used for the second PCR will be about the same size for each primer pair. However, the aliquots can be different sizes as long as, for example, the relative amount of the first PCR that is used for each primer pair is known and the carry-over from first round PCR will not interfere with the second round PCR. Multiple second PCRs can be performed if less than (1/(the number of primer pairs)) is used for each aliquot. For example, if less than or equal to (0.5/(the number of primer pairs)) is used at least 2 second PCRs can be performed for each primer pair, and if less than or equal to (0.33/(the number of primer pairs)) then at least 3 second PCRs can be performed for each primer pair, and if less than or equal to (0.25/(the number of primer pairs)) is used then at least 4 second PCRs can be performed for each primer pair, and so forth. Aliquots can also be used to perform subcloning and standard curve generation as discussed herein.

A typical difference between the first PCR and the second PCR is that the second PCR is typically performed with only one specific primer pair or a subset of specific primer pairs, not the same group of specific primer pairs used in the first PCR. It is understood that when an aliquot of the first PCR is taken, a small amount of the original group of primer pairs is still present in the mixture, because the first PCR mixture was still amplifying at about, for example, 95% the doubling rate which means in part, there was still an excess of the primers, over the amount of product, present in the mixture. These remaining primers do not interfere with the second PCR because the second PCR typically has had an additional amount (amount typically in excess of template) of one of the primer pairs added or has had a related but different primer pair added to the second PCR mixture. Thus, what is typically required for the second PCR is either a) a change in the relative concentrations of at least one of the primer pairs as compared to the other primer pairs in the group of first primer pairs present in the first PCR by adding more of one or more primer pairs to the second PCR, or b) the addition of a new primer pair, not present in the group of first primer pairs, but which is related to, and typically specific for, one of the nucleic acid target products produced in the first PCR. It is understood that the second PCR can also be a combination of a) and b).

Typically the second PCR will be performed with either the same specific primer pair for the specific target nucleic acid molecule or a slightly different specific primer pair for the target nucleic acid molecule. Typically if the second PCR primer pair is slightly different than the related first PCR primer pair, the second PCR primer pair still has the same or similar hybridization regions, meaning that the second PCR will typically hybridize with the same region of the target molecule and target molecule product. What typically will be different is the presence of a sequence or modification that allows for detection of the primer when hybridized to a target nucleic acid. For example, fluorescence detection during real time PCR can occur with any functional technique.

The second round of PCR can also be done with a nested PCR strategy where the second set of primers, used for quantitation, would be used to amplify a region within the amplicon produced in the first round of PCR. This type of system would require that all the primer pairs for one gene would be compatible.

While other methods can also be performed to monitor the amplified products, for example, blot assays, RNAs protection assay, these approaches would only be a semi quantitative approach, as they will not produce absolute copy numbers of template.

The second PCR can be performed using any conditions appropriate for the primer pairs and templates being used. For example, the concentration of the dNTPs or primers or enzyme or buffer conditions can be any concentration that allows the PCR to occur. Typically the concentration of the dNTPs can be between 2.5 and 10 mM each. Typically the concentration of the primers can be between 0.1 and 0.5 μM each. Typically the concentration of the enzyme can be between 1 to 3 units per reaction. Typically the concentration and make up of the buffers is, for example, 1× final concentration out of a 10× stock solution of the manufacturer of the thermal stable polymerase recommended mixture. But it is understood that conditions other than these can also work, and in some cases may be determined after empirical testing.

Any type of thermal stable polymerase can be used. If a hot-start is going to be performed it is preferred that the thermal stable polymerase be of the type that is not functional until an extended period of incubation at a high temperature, such as greater than 90 degrees C.

In general principle, real time PCR uses fluorescence detection, wherein a fluorescent reporter (e.g., fluorescein, FAM, etc.) is coupled to a quencher, for example, DABSYL or Black Hole. During the elongation of PCR, the quencher separates from the fluorescent reporter, resulting in fluorescence. For example, a small nucleotide sequence within a primer sequence can include a fluorescent reporter and a quencher that is sufficiently close to the reporter that no fluorescence emitted. Once the sequence containing the reporter/quencher is incorporated into the PCR product, the quencher is released from the reporter, and the reporter fluoresces. In an alternative approach a short nucleotide sequence, referred to herein as the Z sequence, contains the fluorescent reporter and the quencher. When the uniprimer extends, it recognizes and interacts with the Z sequence in a way that releases the quencher, resulting in fluorescence.

### (5) Generation of the Standard Curve and Analysis of the Second PCR Product

The disclosed methods are designed to allow quantitative analysis of the expression of target nucleic acid molecule, for example, target genes in a given sample. While PCR methods exist to provide accurate information about the doubling rate and amplification activity for a PCR, for example real time fluorescence PCR, the information gained from these types of methods does not provide information as to the exact amount of target nucleic acid starting material in the first PCR or in the sample. The disclosed methods can provide such information. To acquire this information, the information gained in the second PCR about doubling rate and amounts of DNA must be correlated to the starting material used in the first PCR. Typically this is achieved by, for example, generating a representative standard curve for the group of products produced in the first PCR or by generating a standard curve for each individual product in the group of target products from the first PCR. This standard curve will typically relate an absolute amount of DNA to a particular cycle of PCR amplification. Then, the data obtained from the second PCR, for example, the particular cycle that the DNA product reached a certain amount can be placed on the standard curve and an absolute amount of DNA can be determined. The standard curve can be generated in a variety of ways, for example, by taking an aliquot of the first PCR, subcloning the PCR products, amplifying the subcloned products, quantifying the subcloned product using traditional means, such as UV absorbance, and then producing a series of PCRs with varying dilutions of the starting material and performing PCR. Data obtained from these actions will allow a standard curve to be produced which plots, for example, the PCR cycle where the first PCR product

Also, standard curves can be used in the disclosed methods, but other methods to derive absolute copy number of targets, such as analysis using C(t) can also be used.

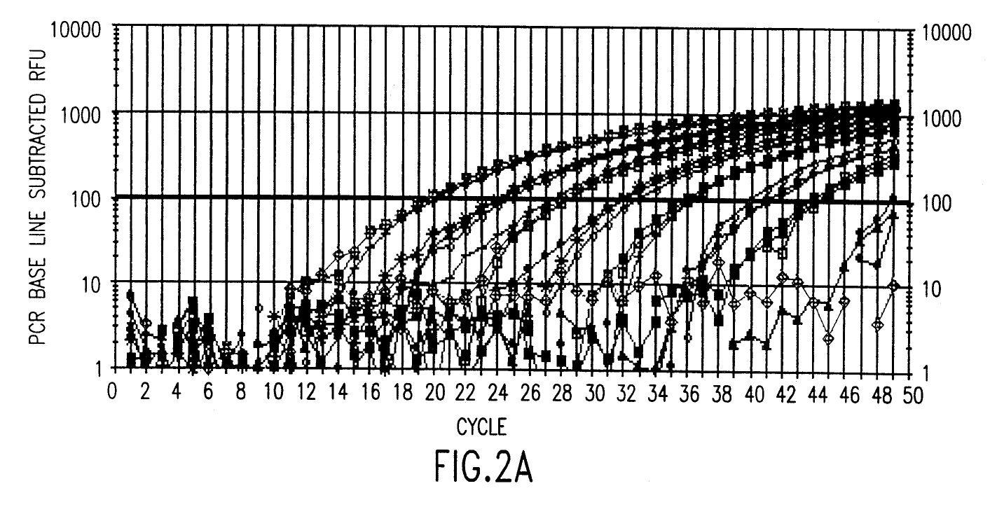

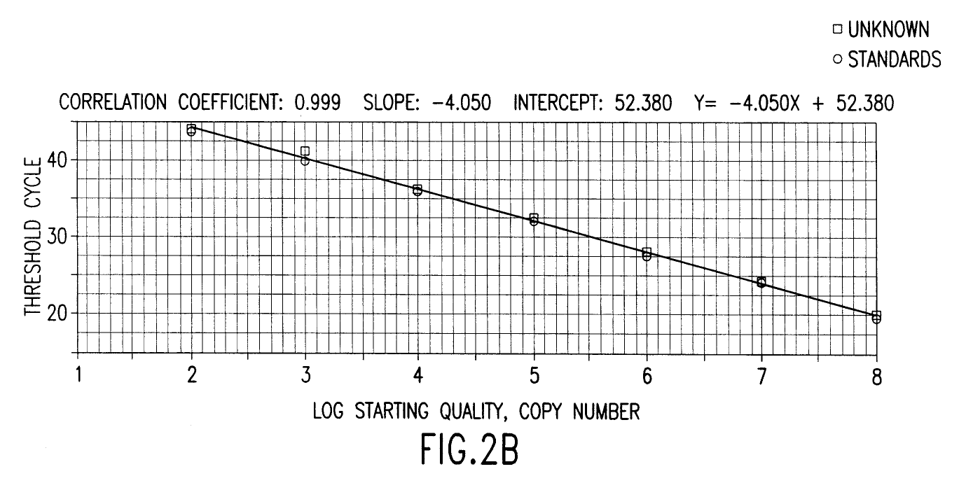

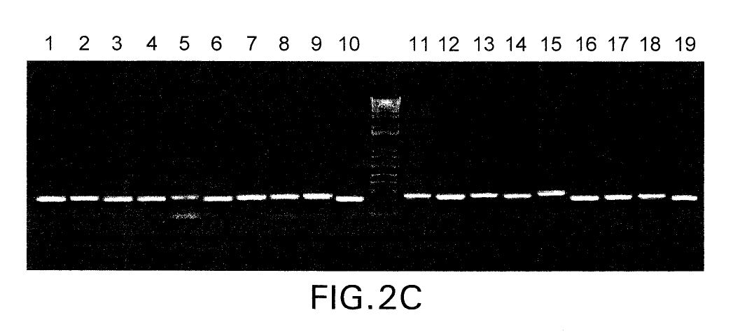

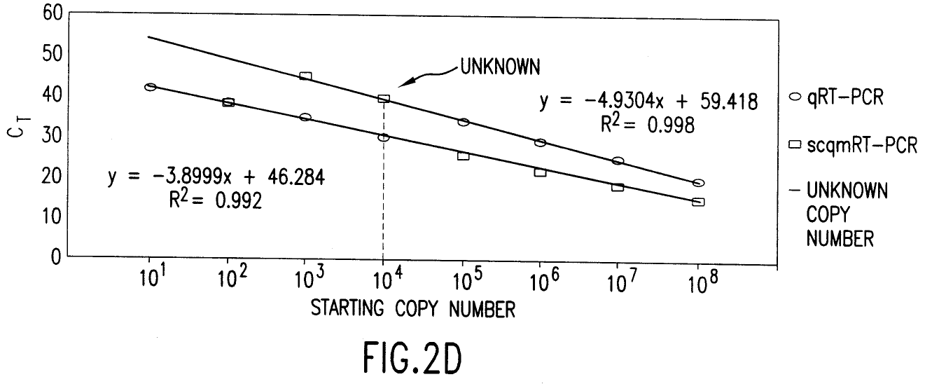

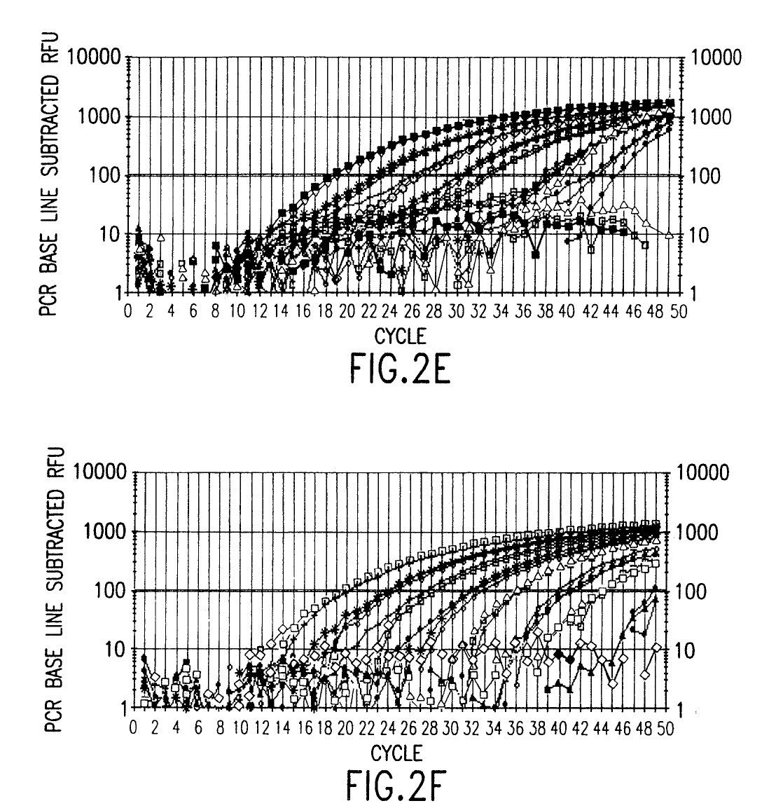

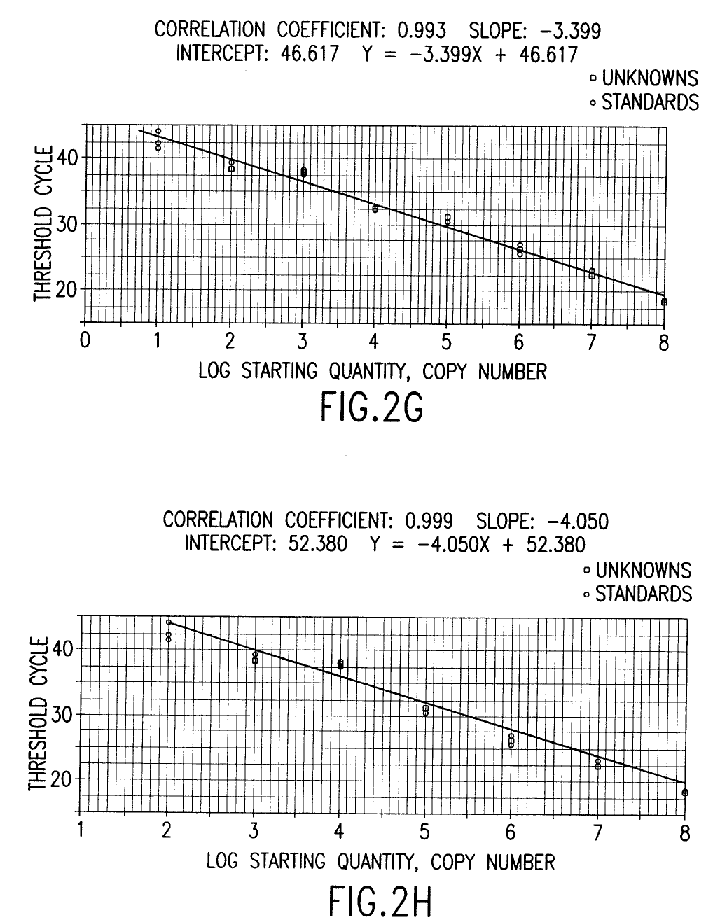

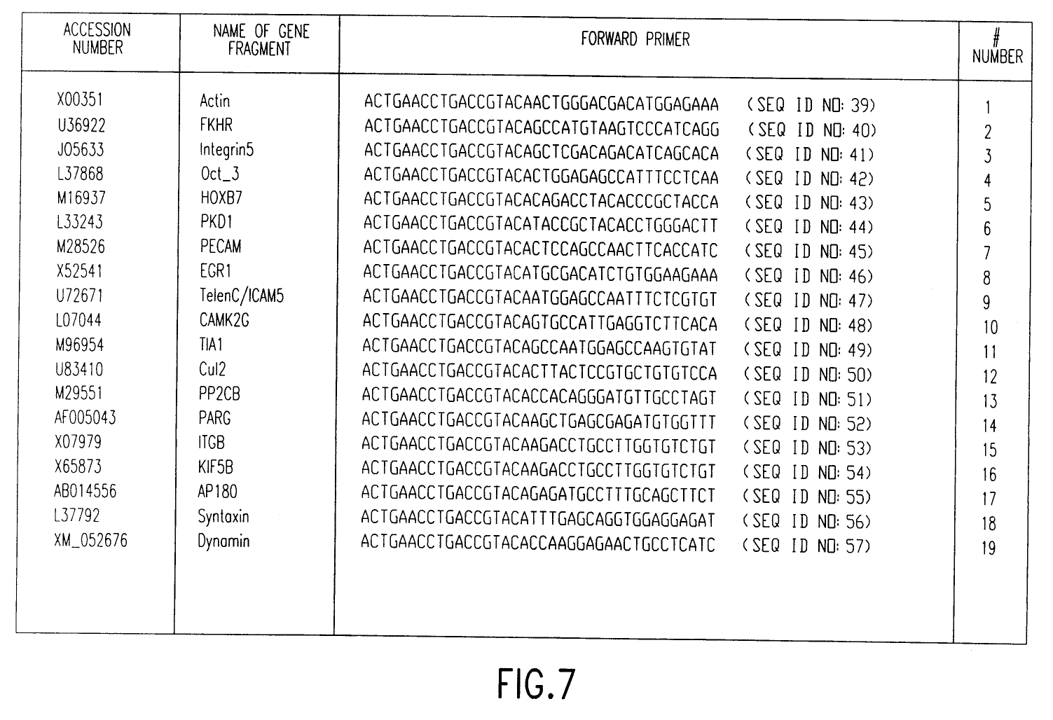

To understand the idea of a standard curve the fundamentals of PCR and in particular real time PCR must be understood. While these concepts are understood by those of skill in the art, a brief, non-limiting discussion is provided here. As discussed herein, PCR is a means of amplifying very small amounts of DNA, often a non-detectable amount of DNA, to levels which can be detected or more easily detected. There are a number of means for detecting target nucleic acid product in a PCR mixture. For example, the products can be detected on an agarose gel which separates the products by size and is detected via UV absorbance, or radioactivity if the PCR is performed with radiolabeled deoxynucleotides for example, or fluorescence if the PCR is performed with fluorophore labeled dNTPS or primers. While these types of protocols can be performed at each cycle of PCR, because there is a manipulation of the sample that must be done to acquire the information, it can be time consuming. Other protocols exist for analysis at each cycle of the PCR without manipulation. This type of protocol is generally termed real time PCR and is typically performed in a thermal cycler that has the capability to analyze the PCR mixture directly, during the reaction process, for example, by directly monitoring a signal generator, such as a fluorphore, in the product. (see, for example, Holland, P. M., Abramson, R. D., Watson, R. and Gelfand, D. H. (1991) Detection of specific polymerase chain reaction product by utilizing the 5′ to 3′ exonuclease activity of Thermus aquaticus DNA polymerase. Proc. Natl. Acad. Sci. USA 88, 7276-7280; Tyagi, S, and Kramer, F. R. (1996) Molecular beacons: probes that fluoresce upon hybridization. Nat. Biotechnol. 16, 49-53; Wittwer, C. T., Herrmann, M. G., Moss, A. A. and Rasmussen, R. P. (1997) Continuous fluorescence monitoring of rapid cycle DNA amplification. Biotechniques 22, 130-138; Rasmussen, R., Morrison, T., Herrmann, M. and Wittwer, C. (1998) Quantitative PCR by continuous fluorescence monitoring of a double strand DNA specific binding dye. Biochemica 2, 8-11; Nitsche, A., Steuer, N., Schmidt, C. A., Landt, O. and Siegert, W. (1999) Different real-time PCR formats compared for the quantitative detection of human cytomegalovirus DNA. Clin. Chem. 45, 1932-1937; Winer, J., Jung, C. K. S., Shackel, I. and Williams, M. (1999) Development and validation of real-time quantitative reverse transcriptase-polymerase chain reaction for monitoring gene expression in cardiac myocytes in vitro. Anal. Biochem. 270, 41-49; and Walker N.J. (2001) Real-Time and Quantitative PCR:Applications to Mechanism-Based Toxicology. J Biochem Molecular Toxicology. 15, 121-27, which are all herein incorporated by reference at least for material related to methods and reagents for performing PCR.) The term “real time” generally refers to the ability to monitor the changing amounts of the target PCR product as the product is being generated, at for example, each cycle of PCR. Regardless of how this monitoring occurs, there is typically a point in real time PCR where the PCR product is just visible over the background detection. In other words, there is a point in time, typically denoted as a particular cycle of PCR, where the starting template has been amplified enough to just barely observe the product. This point is typically called the threshold point or threshold cycle. When a known amount of DNA is used to perform a real time PCR and the threshold cycle for a given dilution of the known starting material is collected, a type of disclosed standard curve can be generated. This type of data produces a curve generated ftom a plot of the amount of DNA (for example copy numbers of DNA) that existed in the starting material vs. the threshold cycle for that amount of DNA. (see FIGS. 2 and 7) An example showing this type of standard curve generation and how it relates to a specific set of target nucleic acids, related to Alzheimer's Disease is shown in the Examples. A number of different illustrations of how the method can be performed are also disclosed herein.

The standard curve can be generated using any set of conditions that produce curve to which the amount of particular PCR can be correlated. For example, the standard curve will typically be a curve that plots the threshold cycle of a PCR vs the log starting quantity, copy number. For example, a standard curve can be generated as follows. Serial dilutions of equimolar concentrations of a plasmid containing the target nucleic acid, the nucleic acid to be amplified and quantified. This typically will occur for each target nucleic acid to be characterized. For example, if 10 or 20 or 50 or 100 genes are to be analyzed at once, then this would typically be performed for each. The dilutions can be set up in any fashion. For example, you could have serial dilutions of 10⁸ to 10¹ or 10¹¹ to 10¹ or 2¹⁰ to 2¹ or 4¹⁵ to 4¹ copies or any dilution. These dilutions can be used as the starting template for a PCR. This PCR can be performed, the products quantified, and since the amount of starting material is known and serially diluted a curve can be generated that provides information about how the particular primer pair amplifies the target nucleic acid sequence, and a curve plotting, for example, threshold cycle vs copy number (or amount etc) of starting material can be generated. It is understood that this type of standard curve generation can be performed in duplicate, or triplicate, etc to increase the accuracy of the curve. It is understood that these reactions can be performed in many different ways and that the curves can be generated using many different techniques.

Any technique is sufficient as long as the technique allows generation of curve which can be used to correlate the amount of DNA in a sample with a known amount of DNA. For example, by generating RNA or DNA to produce a synthetic internal standard, such as a wild-type or a mutant cDNA, to be coamplified with the non-synthetic internal standard and the initial mRNA copy number, thus predicting the mRNA copy number from the ratio of the endpoint wild-type and internal standard PCR products. (Wang, A. M., et al., (1989). Proc. Natl. Acad. Sci. 86, 9717-9721; Becker-Andrë, M. & Hahlbrock, K. (1989).Nucl. Acids Res. 17, 9437-9446; Gilliland, G., et al., (1990). Proc. Natl. Acad. Sci. 87, 2725-2729. all of which are herein incorporated by reference at least for material related to monitoring nucleic acids). An Alternative technique is fluorometrically monitoring the accumulating PCR products and quantifying against an internal standard assessing the relative decrease, for example, in fluorescein quenching by rhodamine after exonuclease cleavage of dual-labeled probes or, for example, by resonance energy transfer of fluorescein to CyS between adjacent probes (FRET principle) or by using other families of cyanine dyes (Holland, P. M., et al., (1991) Proc. Natl. Acad. Sci. 88, 7276-7280; Gibson, U. E., et al., (1996) Genome Res. 6, 995-1001; Livak, K. J., et al., (1995) PCR Methods Appl. 4, 357-362. all of which are herein incorporated by reference at least for material related to monitoring nucleic acids). Another technique is quantifying against a constitutively expressed house-keeping gene by monitoring the fluorescence of a double strand-specific dye at separate product-specific melting temperatures during each cycle (Wittwer, C. T., et al., (1997) BioTechniques 22,130-131, 134-138 herein incorporated by reference at least for material related to monitoring nucleic acids). Alternatively, two closely related mRNAs present in the same sample as internal standards for each other, can be used (Karttunen, L., et al., (1996) Genome Res. 6, 392-403 herein incorporated by reference at least for material related to monitoring nucleic acids).

Another example is where the standard curve is used in conjunction with a technology where the Taq polymerase enzyme cleaves an internal labeled nonextendable probe during the extension phase of the PCR. In this approach, the probe is dual-labeled, with a reporter dye, for example, FAM(6-carboxyfluorescein), at one end of the probe and a quencher dye, for example, TAMRA (6-carboxytetramethylrhodamine), at the other extremity. In this approach, when the probe is whole, fluorescence energy transfer occurs through which the fluorescence emission of the reporter dye is absorbed by the quenching dye. On nuclease degradation of the probe during the PCR, the reporter and quencher dyes are separated, and the reporter dye emission is no longer transferred to the quenching dye, resulting in an increase of reporter fluorescence emission (for example at 518 nm for FAM). (C. A. Heid, et al., Genome Res. 6 (1996), 986-994 herein incorporated by reference at least for material related to monitoring nucleic acids)). Any technology that uses an internal or an external standard would can be used to assess the copy numbers of any mRNA present in the starting material.

One way of analyzing the data of the standard curves is to compare the tlreshold cycle variation, between different data sets. For example, if the threshold cycles between two different curves differs by less than or equal to about 2, 2.5, 2.0, 0.9, 0.8, 0.7.0.6, 0.5, 0.4, 0.3, 0.2, 0.18, 0.16, 0.14, 0.12, 0.10, 0.08, 0.06, 0.04, or 0.02 cycles, the curve can be used to determine the starting material for a given sample. Another way to judge the quality of the standard curve is to look at the correlation coefficient which are understood. For example, the correlation coefficient can be greater than or equal to 0.999, 0.980, 0.970, 0.960, 0.950, 0.940, 0.930, 0.920, 0.910, 0.900, 0.850, 0.800, or 0.750. This level of correlation can occur over at least 3, 4, 5, 6, 7, 8, 9, 10, 11, or 12 orders of magnitude. Another way to assess the quality of the standard curve would be to judge the efficiency of the PCR reaction using the formula y=(10^{1/slope})−1 where y is the efficiency and where, for example, a slope equal to −3.32 would represent a 100% efficiency, a slope equal to −3.40 represent a 97% efficiency, a slope equal to −3.59 represent a 90% efficiency, a slope equal to −4.04 a 77% efficiency. It is also understood that once a standard curve is generated for a particular primer pair and target nucleic acid it can be used to analyze multiple samples, ie it does not have to be generated denovo each time a sample is to be tested.

### b) Specific Illustrations of Using the General Method

### (1) General Examples

As discussed herein there are many different ways to practice the disclosed methods and variations which can be added or subtracted. The following set of illustrations does not represent a comprehensive set of the ways to practice the disclosed methods. The unifying factor between these illustrations is the performance of a first PCR with a group of first primer pairs, a second PCR with less than the full group of primer pairs or a different group of primer pairs, typically only one primer pair, and the comparison of the data obtained in the second PCR to a standard.

### (a) Illustration 1

A target population of lung cancer cells is obtained. 25 genes are determined as target genes and two different primer pairs are obtained for each target gene, a first PCR primer pair and a second PCR primer pair. The primer pairs are determined to be compatible by performing a multiplexed PCR analysis showing that each primer amplifies the target gene. Total RNA is isolated from the population of lung cancer cells using any method, and 0.1 μg of RNA is obtained. The RNA is used to produce a first strand of cDNA using any method. This first strand of cDNA is used in a first PCR which contains the group of 25 first PCR primer pairs which are specific for the 25 target genes. The first PCR is performed for 18 cycles of PCR. The first PCR is then split into 4 sets of 25 aliquots. The first set of aliquots is used to produce the standard curves and is subcloned into a plasmid of choice. The subcloned plasmid is amplified, collected, and quantified. The collected plasmid is then serially diluted so that PCR mixtures containing 10¹, 10², 10³, 10⁴, 10⁵, 10⁶, 10⁷, or 10⁸ copies of the subcloned plasmid are produced. Real time PCR is then performed on these serially diluted plasmid PCR mixtures and the threshold cycle for each is determined. The threshold cycle is then plotted vs. the copy number of starting DNA producing the standard curves for each primer pair. The other three sets of the 25 aliquots are used in the second PCR. Thus, 3 sets of different PCR mixtures, each corresponding to one of the 25 aliquots is made up. Each one of the 25 second PCR mixtures has one of the second PCR primer pairs, which is specific for one of the 25 target genes, added to it and real time PCR is performed. The threshold cycle for each of the 25 reactions is determined and this is done in triplicate because there are 3 sets of 25. This threshold cycle is then correlated to the standard curve produced for the corresponding target gene plasmid and a copy number of the starting material in the second PCR is obtained. This number can then be compared, for example, to the copy number of the other 25 target genes and a quantitative assessment of the relative numbers of the target material in the sample can be obtained, as the amount of material in the starting target sample correlates with the amount of starting material in the second PCR. This data could then, for example, be compared to data obtained from a DNA array analysis of the same 25 target genes from the same target sample.

### (b) Illustration 2

Illustration 2 is related to illustration 1, in that the method is being performed using the same group of 25 target gene first primer pairs and the same 25 second primer pairs. The standard curves have already been generated. Rather than using a lung cancer population of cells, however, a prostate cancer population of cells is targeted. Therefore, just as before, the RNA is isolated, cDNA is made, a first PCR is performed, a second PCR is performed, and then the data is compared to the standard curves to produce a quantitative assessment of the relative quantities of the expression of the target genes in the prostate cancer cell sample. The performance of this method, however, did not require generation of a standard curve de novo, or a new determination of the compatibility of the primer pairs.

### (c) Illustration 3

Illustration 3 is similar to both illustrations 1 and 2. In illustration 3, while the target cell population is still a prostate cancer cell population, in this illustration rather than generating the cDNA library de novo, a commercially available prostate cDNA library was purchased. Thus, this variation of the method only requires the performance of the first PCR, the performance of the second PCR, and the comparison of the data to the already generated standard curve. The isolation of the RNA and the production of the cDNA, as well as the generation of the standard curve and the primer pair determination, are not required.

Disclosed are methods of quantifying gene expression in a target cell population, comprising the following steps 1) performing reverse transcription of the nucleic acid in the target cell population producing cDNA, 2) performing a first PCR with the cDNA producing a first PCR product, 3) performing a second PCR with the first PCR product producing a second PCR product, 4) comparing the amount of the second PCR product to a standard curve, and 5) determining the amount of the second PCR product.

Disclosed are methods of quantifying gene expression in a target cell population, comprising the following steps 1) performing reverse transcription of the nucleic acid in the target cell population producing cDNA, 2) performing a first PCR with the cDNA producing a first PCR product, wherein the first PCR is performed with at least two sets of gene specific primers, 3) performing a second PCR with the first PCR products producing a second PCR product, wherein the second PCR is performed with one set of gene specific primers, 4) comparing the amount of the second PCR products to a standard curve, and 5) determining the amount of the second PCR product, and 6) correlating the amount of the second PCR product to the amount of expression of the corresponding gene in the target cell population.

Disclosed are methods of quantifying gene expression in a target cell population, comprising the following steps 1) performing reverse transcription of the nucleic acid in the target cell population producing cDNA, 2) performing a first PCR with the cDNA producing a first PCR product, 3) performing a second PCR with the product of the first PCR producing a second PCR product specific for each gene of interest analyzed in parallel, 4) comparing the amount of the second PCR product to a standard curve, and 5) determining the amount of the second PCR product, wherein the amount can be determined to absolute copy numbers of template.

The disclosed methods are quantitative for multiple products produced from a single target material. By quantitative it is meant that when the methods are performed the difference between two transcripts can be statistically determined to at least a 10 fold, 9 fold, 8 fold, 7 fold, 6 fold, 5 fold, 4 fold, 3 fold, 2 fold, 1 fold, 0.9 fold, 0.8 fold, 0.7 fold, 0.6 fold, 0.5 fold, 0.4 fold, 0.3 fold, 0.2 fold, 0.1 fold, or at least a 0.05 fold difference. Thus, the present method could determine with statistical significance, a difference between 10 copies of the template of one target transcript and the difference between 30 copies of transcripts, which would be a 3 fold difference.

Disclosed are methods of quantifying gene expression in a target cell population, comprising the following steps 1) performing reverse transcription of the nucleic acid in the target cell population producing cDNA, 2) performing a first PCR with the cDNA producing at least 5 different PCR products, 3) performing a second PCR with the 5 first PCR products in 5 separate PCR mixtures producing at least 5 second PCR products, 4) comparing the amount of the 5 second PCR products to a standard curve, and 5) determining the amount of the 5 second PCR products.

### (2) Specific Disease Targets (Diagnosis)

The disclosed methods can be used to assess specific cell types or cell populations for a particular phenotype or tendency to have a particular phenotype. For example, the disclosed methods can be used to assess the differences between a prostate cancer cell and normal prostate cell or a lung cancer cell and a normal lung cell or an arterial cell from the artery of a subject affected by coronary heart disease and an arterial cell from the artery of a person without coronary heart disease. Use of the disclosed methods in this manner can allow predictions related to the specific phenotype. Different types of target cells or samples can have different groups of primer pairs. Disclosed herein are examples of specific primer pairs that can be useful in the disclosed methods.

### (a) Sets of Primer Pairs

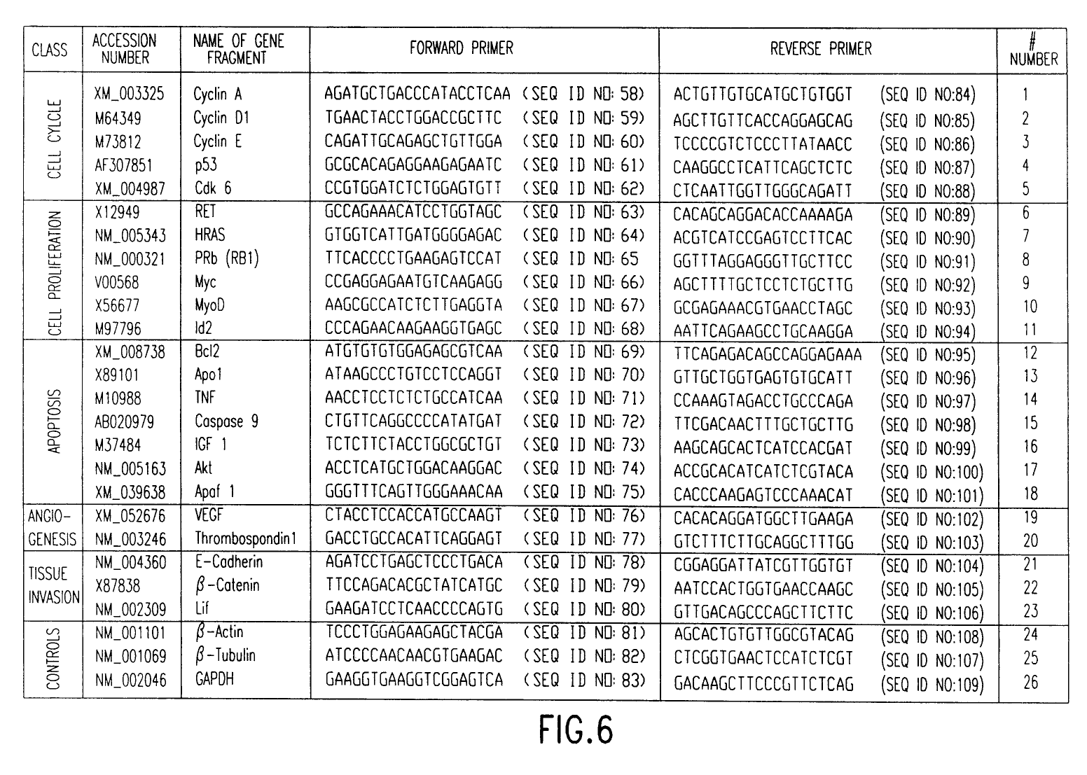

It is understood that any combination of primer pairs that functions as described herein can be produced to analyze any transcript set desired. For example, it is understood that a variety of genes are involved in oncogenic events and that abberent expression of many different genes can occur in many different cancers. The disclosed methods can be used to assay these differences, for example, between different types of cancer cells or between cancer cells and non-cancer cells. An exemplary primer pair for targeting the expression of a variety of genes thought to be involved in oncogenic events is shown in FIG. 6 (SEQ ID NOs: [58-109] (FIG. 6)). It is understood that other primer pairs can be generated.

Primer pairs could be generated and the disclosed methods could be used for a variety of situations and cellular conditions. For example, primer pairs could be generated to analyze, developmental issues, various disease states, stem cells, and cell lineage analysis.

### c) Methods of using the compositions as research tools

The disclosed compositions and methods can be used in a variety of ways as research tools. For example, the disclosed compositions, such as SEQ ID NOs: 1-109 can be used to study the expression patterns in neurons.

The disclosed compositions and methods can also be used diagnostic tools related to diseases such as Alzheimer's and cancer.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

The disclosed compositions and methods can be used to validate oligo-arrays and cDNA Arrays or any other type of DNA diagnostic. The disclosed compositions and methods can also be used to perform single cell quantitative analysis of gene expression.

The disclosed methods can be used for the diagnosis of a variety of diseases. Any disease which is associated with the differential expression of or accumulation of or degradation of the mRNA of one or more genes can be assayed or diagnosed using the disclosed methods. For example, neurodegenerative diseases such as Alzheimer's Disease, Amyotrophic Lateral Sclerosis, Ataxia, Cerebral Palsy, Dysautonomia, Epilepsy, Huntington's Disease, Hydrocephalus, Lewys body Disease, Meningitis, Olivopontocerebellar Atrophy, Parkinson's Disease, Rett Syndrome, and Tourette Syndrome or cellular degenerations arising from Autonomic Nervous System, Chromosomal Disorder, Chronic Fatigue Syndrome, Chronic Pain Syndromes, Congenital Anomalies, Cranial Nerve Diseases, Dementia, Demyelinating Diseases, Headaches, Infections, Movement Disorders, Muscle Diseases, Neoplasms, Neurocutaneous Syndromes, Neurologic Manifestations, Neurotoxicity Syndromes, Ocular Motility Disorders, Peripheral Nervous System, Pituitary Disorders, Porencephaly, Sleep Disorders, Spinal Cord, Stroke, Trauma and Injuries, can be diagnosed using the disclosed methods. Cancer can also be diagnosed using the disclosed methods. Any cancer that is associated with the differential expression accumulation, or degradation of the mRNA of one or more genes can be diagnosed. For example, Bladder Cancer, Breast Cancer, Cervical Cancer, Colorectal Cancer, Uterine Cancer, Hodgkin's Disease Cancer, Kidney Cancer, Adult Acute Myelogenous Leukemia, Acute Lymphocytic Leukemia, Adult Chronic Myeloid Leukemia, Small Cell Lung Cancer, Non-Small Cell Lung Cancer, Multiple Myeloma, Non-Hodgkin's Lymphoma, Oral Cancer, Ovarian Cancer, Pancreas Cancer, Prostate Cancer, Melanoma, and Testicular Cancer, cancers can be diagnosed.

The methods can be used to analyze the expression of gene patterns in any cell type in which differentiation expression or accumulation or degradation of mRNA occurs. For example, the disclosed methods can be used to look at the differences in mRNA in cells in different stages of the cell cycle, cells in different stages of learning, cells from different phenotypic donors, etc. For example, in the context of neurobiology, the differential expression, accumulation, or degradation of genes, the cell heterogeneity could be observed within the aging and/or degenerating brain.

Also disclosed are methods wherein the disclosed quantitative multiplex PCR methods are coupled to immunocytochemistry. For example, the differential expression, accumulation, or degradation, of the mRNA between two different cells or cell types can be determined wherein the different cells or cell types can also be determined to be different by immunocytochemistry. For example, diseased neurons could be analyzed at the single cell type level, by for example, screening for the neurofibrillary tangles marker, and comparing the expressed mRNA in the neurons positive or negative for the marker.

In the context of early molecular diagnosis of diseases, this technology can serve as a basis to statistical analysis leading to the diagnosis. This technology can be used as a validation and/or an alternative approach to the aRNA amplification technique that is currently used for blood tests. Still in the context of diagnosis, currently single biomarkers are tentatively used to early specify the state of a disease. These approaches are not used on a daily basis as contradictory results arise (for reviews on this topic see Mulder C, Scheltens P, Visser J J, van Kamp G J, Schutgens R B (2000) Genetic and biochemical markers for Alzheimer's disease: recent developments. Ann Clin Biochem 37:593-607. Cowan L D, Leviton A, Dammann O (2000) New research directions in neuroepidemiology. Epidemiol Rev 22:18-23.). The disclosed methods, serving as a basis for canonical analysis or principal component analysis, will avoid theses disadvantages as numerous transcripts can be analyzed in parallel in a sensitive and reproducible way that is complementary to technologies such as aRNA, single-cell mRNA phenotyping, multiplex analysis, the Belyavsky method, PolyAPCR, and TPEA. Finally, in the context of functional genomics, the quantitative multiplex PCR can become a must in term of validation of large cDNA and oligo-arrays. As a matter of fact, the limitations of sensitivity and reproducibility in the latter methods are more and more recognized. These limitations are addressed with the disclosed methods.

### 2. Compositions

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular primer pair is disclosed and discussed and a number of modifications that can be made to a number of molecules including the primer pair are discussed, specifically contemplated is each and every combination and permutation of the primer pair and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### a) Primers

The disclosed rely on primers for extension and amplification of particular DNA products. In the first PCR of the disclosed method, the primers that make up the group of first PCR primer pairs are added in a mixture. Typically the mixture is about an equimolar mixture of the primer pairs. Disclosed are compositions that comprises the mixtures of primers to be used in the first PCR of the disclosed method. These compositions will be mixtures of different primer pairs. For example, disclosed are compositions that comprise the nucleic acids set forth in SEQ ID NOs:1-109 (FIGS. 6, 7, and 8). Also disclosed are compositions comprising the nucleic acids set forth in SEQ ID NOs:1-109 wherein the nucleic acids are about equimolar to each other. Also disclosed are compositions that comprise mixtures of primers that hybridize to primers sets as described in FIGS. 6, 7, and 8.

### b) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

c) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

d) Nucleic acids

There are a variety of molecules disclosed herein that are nucleic acid based, including for example nucleic acid primers, for example, SEQ ID NOs: 1-109. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U.

(1) Nucleotides and related molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and

2-thiocytosine, 5-halouracil and cytosine, 5-propynyl uracil and cytosine, 6-azo uracil, cytosine and thymine, 5-uraeil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂), OCH₃, —O(CH₂), NH₂, —O(CH₂). CH₃, —O(CH₂), —ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₁, SO₂ CH₃, ONO₂, NO₂, N₃, NH₃, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### (2) Primers and Probes

Disclosed are compositions including primers and probes, which are capable of interacting with a variety of nucleic acid molecules, such as gene transcripts and genes as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner.

The size of the primers or probes for interaction with the target nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22,23,24,25, 26,27,28,29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a target nucleic acid primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the target nucleic acids typically will be used to produce an amplified DNA product that contains a region of the target nucleic acid that is between 100 and 350 nucleotides long. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### e) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

### f) Computer Readable Mediums

It is understood that the disclosed nucleic acids and proteins can be represented as a sequence consisting of the nucleotides or amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Also disclosed are computer readable mediums in which standard curves, to be used as disclosed herein, for specific groups of primer pairs, are stored or retrieved for analysis to a particular set of data.

Disclosed are computer readable mediums comprising the primers and information regarding the primers set forth herein.

### g) Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

The kits can contain groups of primer pairs for both the first PCR and the second PCR. The kits can also contain information, for example, about standard curves that are specific for the included groups of primer pairs that are contained within the kit. The kits can contain any of the reagents needed to perform the various forms of the methods disclosed herein.

For example, a kit could contain primers set forth in SEQ ID NOs: 1-109 as well as the primers having the universal beacon sequence attached to SEQ ID NOs: 1-109 as well as the information about the standard curve made for each. The kit would not need to contain any of the reagents as these can be obtained in other ways. In a particular variation of this type of kit, the group of first PCR primer pairs could be in a single tube, ready to be added to a PCR mixture.

### h) Compositions with Similar Functions

It is understood that the compositions and methods disclosed herein have certain functions, such as allowing for multiplex analysis of nucleic acid sequences using PCR. Disclosed herein are certain structural requirements for performing the disclosed functions or steps for performing the disclosed methods, and it is understood that there are a variety of structures or steps which can perform the same functions which are related to the disclosed structures and steps, and that these structures will ultimately achieve the same result, for example stimulation or inhibition allowing multiplex analysis of nucleic acid sequences using PCR.

### 3. Methods of Making the Compositions

The compositions disclosed herein and the compositions necessary to perform the disclosed methods can be made using any method known to those of skill in the art for that particular reagent or compound unless otherwise specifically noted.

### a) Nucleic acid synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### B. TERMS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“Doubling rate” is a term that is used herein, in the context of PCR, to refer to the rate at which a given product produced by a given primer pair under a given set of conditions is amplified in a PCR reaction. In theory, each cycle of amplification that takes place in a PCR reaction (melting, annealing, and extension) produces exactly 2 times the template DNA, i.e., it doubles the template DNA. In practice, however, amplification is never “perfect” and actual amplification occurs at a rate slightly less than “2 times per cycle.” The highest rate of amplification that occurs for a given PCR product under a given set of conditions is termed the “doubling rate” of the reaction and it is understood that it can be less than “double” for example, 1.92 or 1.83. The doubling rate, thus represents an approximate upper level of amplification for a given set of reagents and conditions.

“Optimal PCR amplification” refers to the condition that exists when each cycle of PCR is still amplifying at about the doubling rate for the particular PCR product. It is understood that as PCR product increases in a PCR mixture and the PCR primers decrease in a PCR reaction mixture, the efficiency of amplification decreases below about the doubling rate for the particular product and conditions. When this occurs the reaction is said to no longer be undergoing optimal PCR amplification.

Throughout this application, various publications are referenced. The disclosures of these publications in their entireties are hereby incorporated by reference into this application in order to more fully describe the state of the art. The references disclosed are also individually and specifically incorporated by reference herein for the material contained in them that is discussed in the sentence in which the reference is relied upon.

A primer pair is used herein to refer to a forward and a reverse primer that are designed to amplify a specific target nucleic acid. A primer pair can also be referred to a primer set and a primer set can be referred to a primer pair.

A set of primer pairs refers to at least two primer pairs, which are designed for two different target nucleic acids. Typically this means two different nucleic acid targets, from for example two different genes, but it could also be two different primer pairs designed to amplify two different isoforms, for example, of the same expressed gene product.

Copy number refers to the number of copies of something. For example, the copy number of a particular nucleic acid refers to the number of copies of that particular nucleic acid exist, in a sample, or a tube, or situation, for example. The copy number can be determined as disclosed herein, and can also be translated into for example, the number of grams of a particular composition or the number of moles of a particular composition as disclosed herein and understood.

### C. EXAMPLES

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1 Validation of Single Channel Quantitative Multiplex RT-PCR for Large Numbers of Gene Products and its Use in the Field of Alzheimer's Disease Research

### a) General overview of design

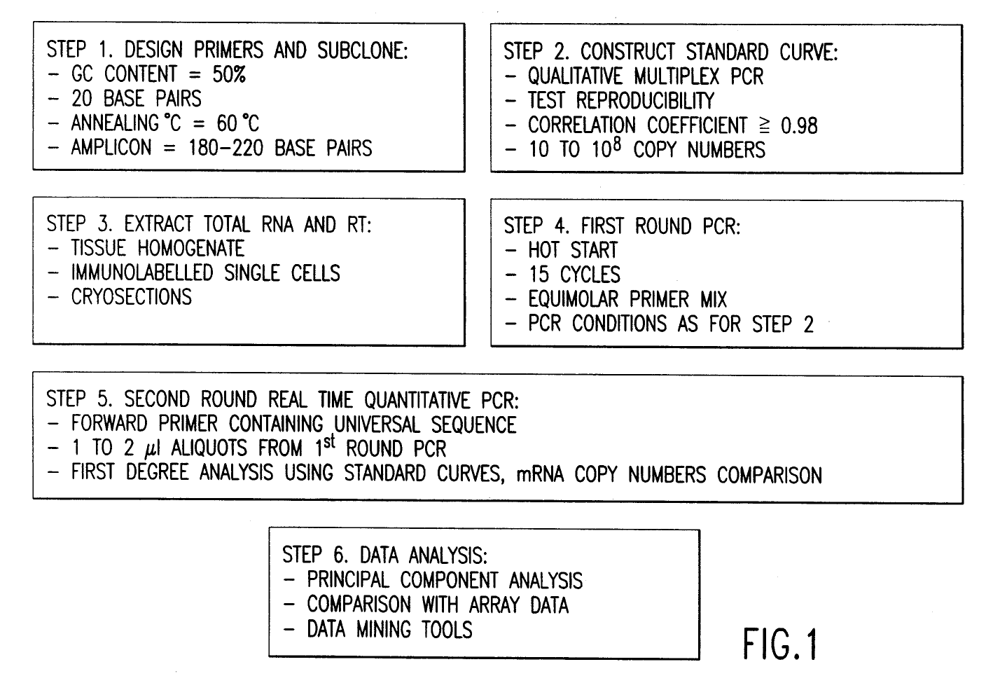

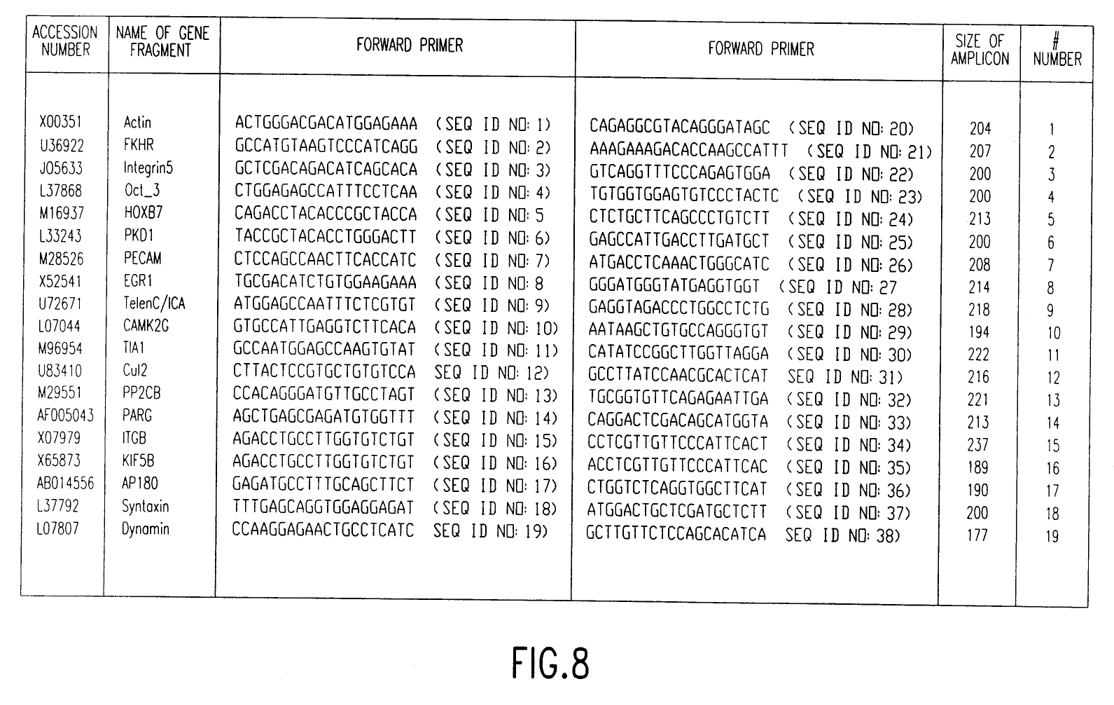

The overall view of an example of the disclosed methods is outlined in FIG. 1. Typically, after homogenization of the tissue, total RNA from superior frontal gyrus was extracted from dry ice or isopentane frozen tissue using an RNA extraction kit (Qiagen). RNA extracted from these homogenates and alternatively tissue scraped from fresh frozen sections that have been fixed with acetone and counterstained with Hematoxylin was reverse-transcribed in a final volume of 20 μl using Sensiscript reverse transcriptase (Qiagen) in the manufacturer's buffer containing the appropriate concentration of dNTP's, RNAse inhibitor (Promega), NVd(T)'s and 1 μg of total RNA. The reaction took place at 37° C. for 12 hours before storage at −20° C. Multiplex Real-Time Quantitative RT-PCR analyses for β-actin, FKHR, Intergrin, Oct3, HOXB2, PKD1, PECAM, EGR1, TelenC, CAMK2G, TIA1, CuI2, PP2CB, PARG, ITGB, KIF5B, AP180, Syntaxin and Dynamin mRNAs were performed using the iCycler instrument and software (Biorad). 3 sets of primers (2 forwards and 1 reverse) were designed to specifically amplify between 177 and 237 base pairs for the genes of interest and were synthesized by Gibco (the primers are shown in FIG. 7 and FIG. 8). One of the forward primer pair contained a “universal sequence” to be used in the second round of amplification (see below). The sequences of the PCR primers and the universal sequence that were used for each gene are shown in FIG. 7 and FIG. 8. The fluorescence probe used for the second round of PCR were labeled with a reporter, such as (FAM=6-carboxy-fluorescein) and a quencher dye (DABSYL=4-(dimethylamine)azo benzene sulfonic acid). The principle of Multiplex Real-Time Quantitative RT-PCR is described herein and typically consists of 2 rounds of PCR. The disclosed methods do not need a specific reporter for each gene of interest. After the reverse transcription, a first round of PCR is performed with 1 μl from the RT. In this round, all the primers of interest are mixed together, typically in an equimolar concentration and in a final volume of 100 μl, with the appropriate concentration of HotStarTaq polymerase (Qiagen) in the manufacturer's buffer containing dNTP's and the 20 μl of RT. After 15 minutes at 95° C., each of the next 15 cycles consisted of 20 seconds of denaturation at 95° C., 20 seconds of annealing at 60° C. and 20 seconds of elongation at 72° C. A final step of elongation at 72° C. for 10 min was performed. For the real-time quantitative second round of amplification, typically an aliquot of 1 μl from the first round was mixed on ice in a total volume of 25 μl with each time the forward specific primer containing the universal sequence and the reverse primer specific for every gene of interest with the appropriate concentration of Taq polymerase (Qiagen), the universal primer (Intergen), and dNTP's. A standard curve is then constructed respectively for each gene of interest. Before doing this, each amplicon was cloned into pGEM®-T Easy vector (Promega). The plasmids generated are quantified using a spectrophotometer (Pharmacia) and diluted sequentially to be used as known starting material for plotting standard curves. These curves can then be used to analyze the starting amount of gene copies for each unknown template, based on its specific threshold cycle.

### b) Results

### (1) Experimental Design of scqmRT-PCR

The overview of the strategy used to analyze numerous transcripts in parallel is outlined in FIG. 1. The first step was to design and validate a set of primers that would share the same PCR conditions. (Ruano et al., 1995). Common parameters included annealing temperature (60+/−0.5° C.), GC content (50+/−5%) and amplicons with 180 to 200 base pairs. Typically, the longer the PCR products the more the background increased. These features were essential to allow all the multiplex PCR reactions to occur at maximum efficiency. Construction of a series of standard curves took place after primer design. To achieve this step, it each candidate was subcloned into a plasmid vector. To do so, a regular qualitative multiplex RT-PCR was performed (data not shown). This step not only allowed subcloning the candidates of interest but also provided a quality test for the primer pairs. In other words, any incompatibility among the primers such as inter-complementarity or self-complementarity could be detected at this point and relevant primers could be redesigned. The specificity of each primer pair was also tested through this step. This “empiric” quality control was found to be less time consuming and more reliable than other controls using primer design software, although this can be performed also. Once our targets had been subcloned, standard curves for each gene of interest were constructed.

The third step (step 3 in FIG. 1) was concerned with sample preparation. Total RNA (Qiagen, Rneasy® Midi Kit) was extracted from samples following the manufacturer's instructions and reverse transcribed into a first strand cDNA. Once the first strand cDNA had been synthesized, a first round of PCR was performed. This first round of PCR was performed with 1 μl of the RT (step 4). In this round, all the primers of interest were mixed together in an equimolar concentration and the final volume was 100 μl, with the proper concentration of dNTP's, enzyme and appropriate mix (see material and methods). In this example, the first round was limited to 15 cycles to guarantee that even the most abundant messages such as β-actin were still within a linear range of amplification when starting with 1 μg of total RNA. The PCR conditions of this first round were the same as in the previous “regular” multiplex RT-PCR (step 2) for subcloning and quality control. The next step (step 5) consisted of a series of single channel real time quantitative PCR reactions. To achieve this step, an aliquot of the first round PCR (1 μl) was mixed on ice in a total volume of 50 μl with each specific forward primer tagged with a universal sequence (Intergen) at 5′ and the reverse primer specific for every gene of interest (Nazarenko et al., 1997; Nuovo et al., 1999; Winn-Deen, 1998). Appropriate concentrations of enzyme and reagents necessary for the reaction were added to the solution. Reactions for all the genes of interest were carried out in parallel. This second round of PCR was performed in a real time quantitative thermocycler (iCycler, Biorad) and quantitation of the fluorescent emission was recorded during each cycle of PCR. The copy number for each gene of interest was then calculated based on threshold cycles using the corresponding standard curve. Thus, 19 quantitations have been performed in parallel from the initial first strand cDNA (step 3). The next step (step 6) consisted of data analysis. Several analyses can be performed to achieve different goals. Examples will be presented in the next sections.

### c) Validation of scgmRT-PCR

As a first step toward the validation of scqmRT-PCR, 19 candidate targets (FIG. 8) were subcloned in cloning vectors (pGEM®-T Easy Vector System, Promega), but any subcloning vector can be used

The specificity of the primers was further confirmed by sequencing each inserted clone of interest. Then, with plasmids containing each target gene, the procedure in FIG. 1 was carried out (steps 2 to 5). 8 serial dilutions of equimolar concentrations of the 19 plasmids ranging from 10⁸ to 10¹ copies were used as a template for the first round of amplification. This allowed the parallel processing of these 19 inserts at different concentrations and allowed the construction of 19 different standard curves from the same aliquot, each time with a different copy number in triplicate. An illustration of the uniformity of the amplifications is shown in FIG. 2A. Analysis of the triplicate repeats led to standard deviations of threshold cycle number ranging from 0.05 to 0.18 cycles (typically about 0.1 cycles) within the dilution series used (In other experiments the number of cycles was not more than 0.4 cycles see FIG. 2E). By using all data collected in this experiment and combining these data with appropriate selections for baseline cycles and threshold, the final result for 24 wells (i.e. 8 dilutions×3 triplicates) was a mean threshold cycle of 29.6 and a standard deviation of 0.21 (SD=0.7% of the mean). Each of the 19 background-corrected data were brought down to the PCR baseline to form standard curves as illustrated in FIG. 2B, which shows a representative example of the amplification linearity that can be achieved with scqmRT-PCR. The standard curve correlation coefficients ranged between 0.999 and 0.980 over a range of 8 orders of magnitude. This range of correlation coefficient is in accordance with what can be obtained using regular quantitative RT-PCR. After each second round of PCR, a 1% agarose gel was run to check for specificity of the amplifications (FIG. 2C). This step is important in scqmRT-PCR as one wants to minimize the chance that nonspecific amplification or contamination arising from another set of primers has occurred. The sensitivity of scqmRT-PCR allows reproducible amplification of starting material containing 10-100 copies of transcript (10 copies as shown in FIG. 2B). In some instances the threshold cycle was reached with single copy template (data not shown). This amount of starting material is equivalent to less than 1 pg of nucleic acid, which is compatible with the sensitivity required for single cell transcript analysis. This level of sensitivity makes the procedure compatible with a large range of applications and reduces by several orders of magnitude the amount of starting material necessary for quantitation over that required for arrays or Northern blots. To further validate scqmRT-PCR the results obtained were compared following regular quantitative RT-PCR and scqmRT-PCR using several targets. FIG. 2D illustrates the results obtained. There, one dilution was set (104 copies in the example provided) as an “unknown” concentration in the thermocycler settings. The first observation was that the absolute value of threshold cycles was significantly reduced using scqmRT-PCR (p<0.001) when standard curves were constructed. This did not affect the calculation of the accurate copy number of starting material, since the pre-amplification was kept in the linear range, thus not changing the original relationship between threshold cycles and log of copy number. It should be noted that separate standard curves for regular quantitative RT-PCR were constructed. Yet, using both approaches, the same copy number of starting material was obtained. Therefore, our technology gives the same result in term of absolute quantification relative to plasmid when compared to more traditional approaches.

### d) Application of scqmRT-PCR in the Context of Alzheimer's Disease

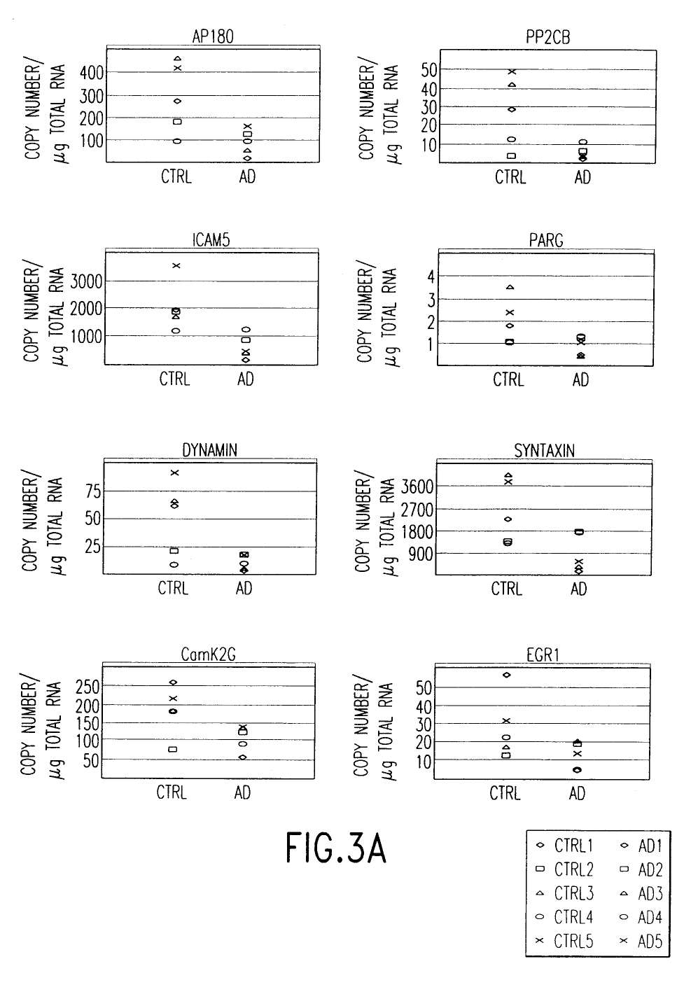

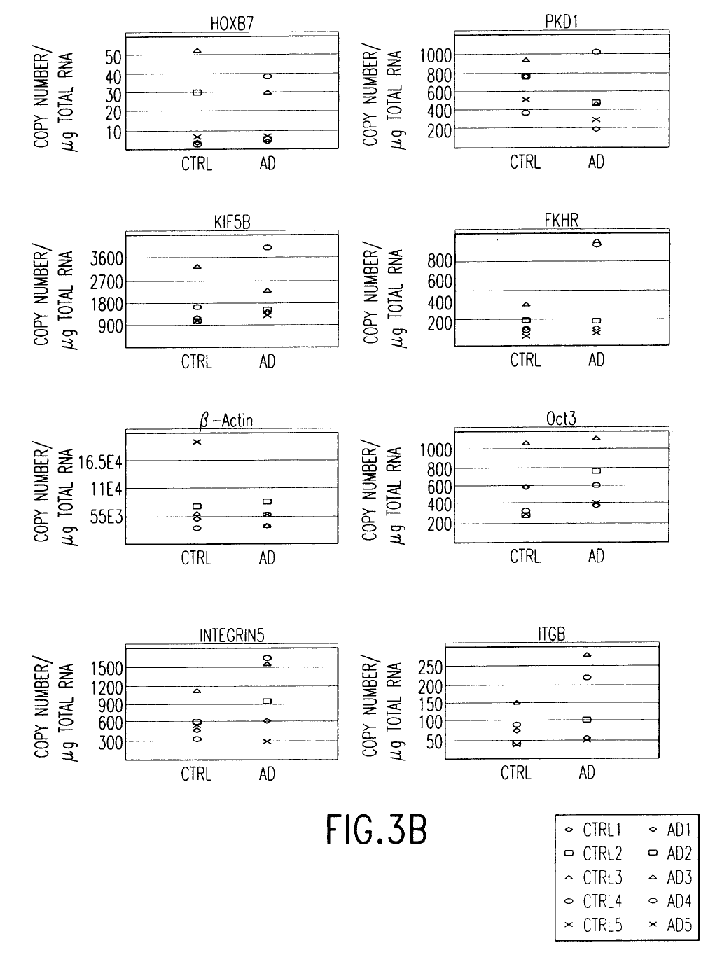

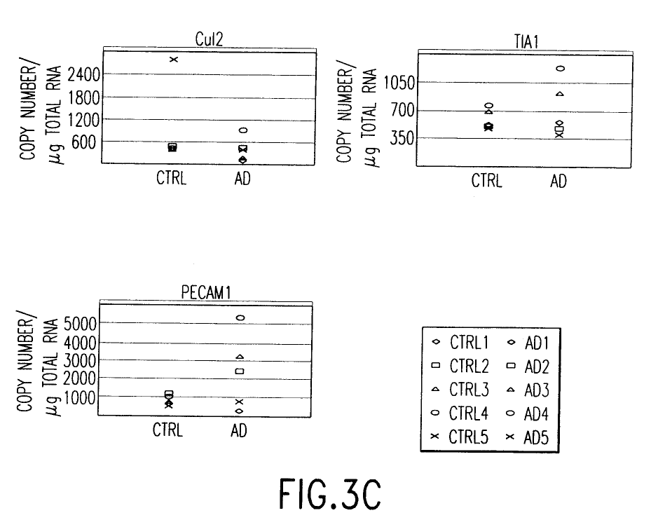

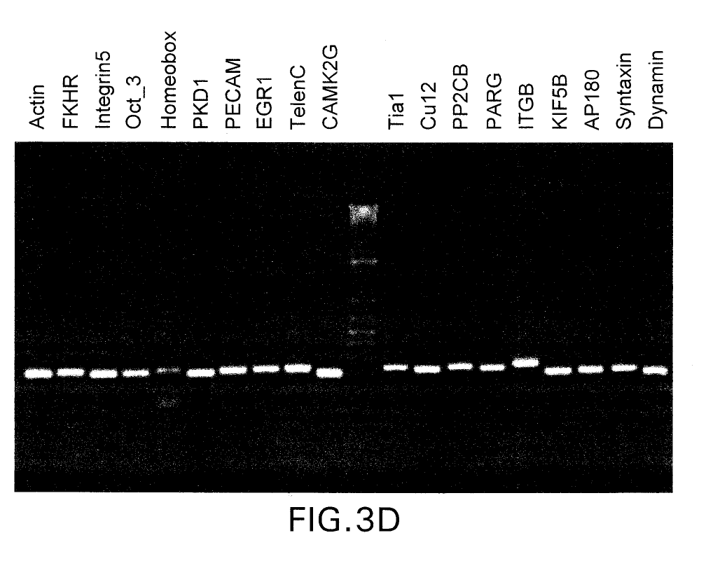

After these validation steps, it was important to demonstrate that the approach can be applied in a biological context. To do so, the expression of 19 targets in AD (see FIG. 7 and FIG. 8) were investigated. For comparative purposes, these targets were selected based on results obtained with oligo-arrays that will be discussed below. After adequate proteinase K (Roche) and DNAse I (Promega) digestions, the total RNA was extracted following the manufacturer instructions. Comparison of results obtained from total RNA vs. mRNA showed no difference in data reproducibility (data not shown). Consequently, subsequent preparations used total RNA. 5 AD cases, 3 age-matched controls (described as “controls” in the text), and 2 cases whose autopsy report met the neuropathological criteria of AD of the Reagen Institute (Gearing et al., 1995; Mirra et al., 1994) but had no clinical signs of dementia (Retrospective Clinical Dementia Rate: 0) were used. These 2 cases are described as “intermediate” throughout the text. The AD cases satisfied both clinical and neuropathological criteria for AD (Gearing et al., 1995). FIG. 3 summarizes the copy numbers per μg total RNA of 19 genes obtained from the 10 cases studied. Each measure was in triplicate (SD is plotted but is too small to be visible). Due to the inherent variability among human subjects, it was found to be more informative to present the data with each individual described separately rather than grouping the results as “means of controls or means of AD”. The presentation of individual data provides information that may otherwise be obscured. The data show a clear separation between control and AD cases for a number of candidate messages (FIG. 3A). Controls (which did not include the two “intermediate” cases) always had a higher mRNA copy number per μg total RNA for 7 candidate genes studied: AP180, PP2CB, Dynamin, Syntaxin, PARG, CAMKG, ICAM5 (FIG. 3A). A majority of these targets are related to the dendritic or synaptic apparatus, which has been proposed to be affected early in AD (Maccioni et al., 2001; Minger et al., 2001; Scheff et al., 2001). Moreover, the expression pattern of the 2 intermediate cases was more similar to the AD group than to the control group (FIG. 3A). Within the above 7 messages, it was also observed that the AD population, which is representative of “late” AD stages both clinically and morphologically, was more homogenous in terms of copy numbers than compared to the control and intermediate cases, similar to what has been observed in previous studies (Chow et al., 1998). However, the greater heterogeneity in the cases representing control plus intermediate is almost entirely due to intermediate cases falling among the AD values. Thus, if the cases that were clinical controls but neuropathological AD were excluded from the more strictly defined control population, then the control cases were not more heterogeneous than AD cases. Egr-1 represents an exception to these comments since controls were more heterogeneous and overlapped with AD values (FIG. 3A). Another set of genes, including HOXB7, PKD1, β-Actin, Oct-3, KIF5B, FKHR, Intergrin-β5 and ITGB showed a heterogeneous distribution of copy numbers and extensive overlap of both groups (FIG. 3B). In the case of the endogenous endonuclease TIAL1 (Kawakami et al., 1992) and the acute inflammatory response protein PECAM1 (Newman et al., 1990) the control population and the intermediary cases were more homogenous than the AD group. The hypoxia induced mRNA regulator CUL2 (Pause et al., 1998; Pause et al., 1997) seemed to belong to this group with one noticeable exception in the control population (control case 5). FIG. 3C illustrates a comparison of gene expression in absolute values between age matched controls and AD cases. Standard deviations were higher within the age matched control group compared with the AD cases, revealing the higher heterogeneity of the age matched control group (FIG. 3C). β-actin was used as a house keeping gene for normalization of the data between the 2 groups (FIG. 3D). Consistent changes in relative expression were observed. A 9 fold change between β-actin in aged-matched controls and AD was found.

### e) Application of Principal Component Analysis

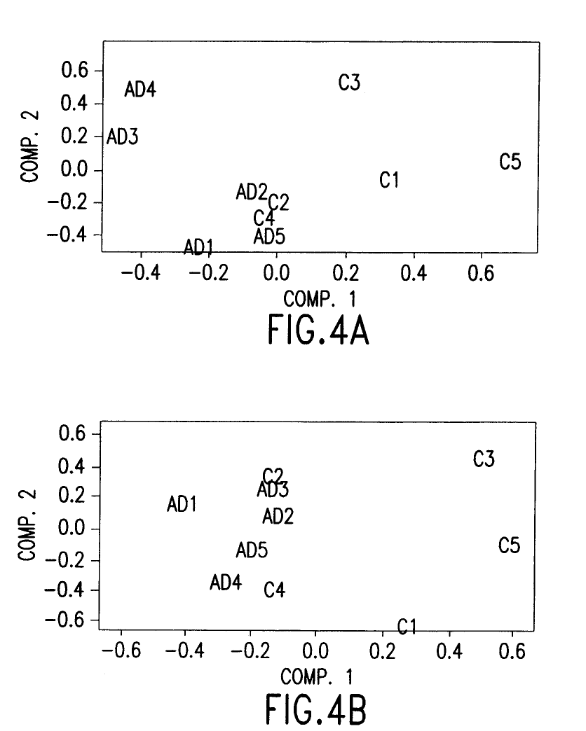

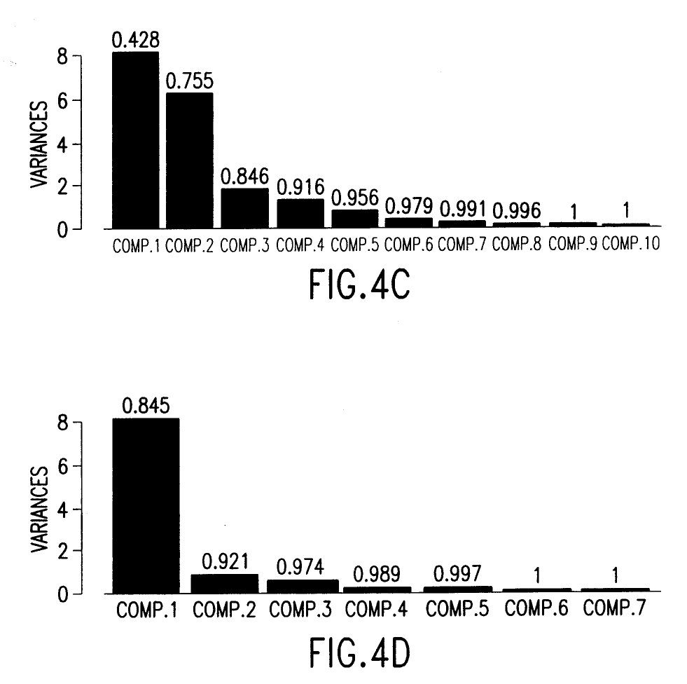

Principal component analysis (PCA) was used to reduce the dimensionality of our data set and to extract further meaningful biological information. First, the entire set of genes was used to perform PCA and a 2 dimensional plot of the first 2 principal components was constructed (FIG. 4A). This analysis showed clustering of cases according to their disease status. In particular, the intermediary cases were positioned closer to the AD cluster than to the control cluster. The first 2 components were sufficient in this analysis to account for 75.5% of the variance among our candidates (FIG. 4C). The messages that contributed heavier weights to component 1 were Dynamin, AP180, ICAM5, PP2CB, Syntaxin and Actin. The messages that contributed heavier weights to component 2 were PKD1, KIF5B, HOXB7, Integrin5, ITGB and FKHR. The 7 genes related to the dendritic and synaptic apparatus (see FIG. 3A) were then selected and PCA was performed on this set of candidates. Using this collection of genes a more pronounced clustering of the AD cases and again the intermediary cases were closer to AD cases than to controls (FIG. 4B) was observed. Here, the first component accounted for 78.5% of the variance among our candidates (FIG. 4D). Addition of the second component increased the variance accounted for to 92.3%. The fact that this set of messages allowed a clear separation between AD and control suggest that they could be used to separate AD from control groups with low probability of error. Furthermore, they could be used in any post-mortem situation where the final diagnosis of AD is difficult (Gearing et al., 1995). Moreover, this set of results can lead to further investigations about the connection of these genes to the cell biology of the disease. Altogether, the data presented in FIGS. 3 and 4 demonstrate that scqmRT-PCR can be used in a biological paradigm where transcript populations are of interest.

### f) Affymetrix Samples Compared with scqmRT-PCR

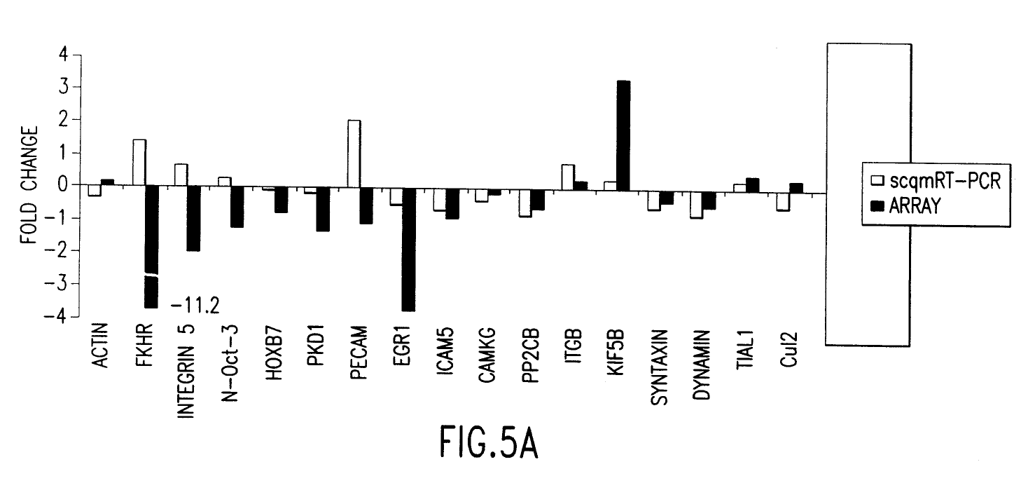

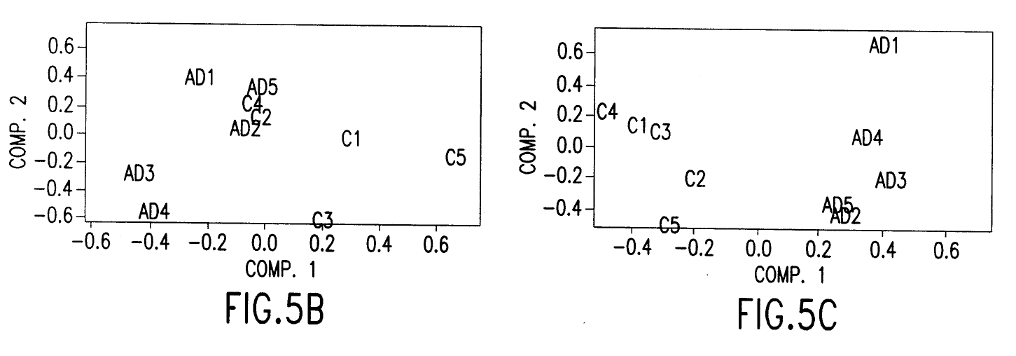

Another use of scqmRT-PCR is to validate oligo-arrays or cDNA arrays. En masse identification of the mRNAs differentially expressed between controls and AD cases was achieved using Affymetrix Human U95 oligonucleotide microarrays. Total RNA extracted from the same cases used for the rest of this study were hybridized and analyzed following published procedures (Golub et al., 1999). The fold changes for each target between age-matched controls and AD cases (FIG. 5A) were then compared. The synaptic vesicle endocytosis AP180 (Mao et al., 2001) was excluded, as this gene is not represented on the oligonucleotide arrays. In 7 (41%) out of the 17 targets a discrepancy between the 2 techniques in terms of a trend greater than one fold change was noticed. These 7 targets were FKHR (Anderson et al., 2001), Integrin 5 (Reynolds et al., 2002), Oct 3 (Schreiber et al., 1993), PKD 1 (European Polycystic Kidney Disease Consortium, 1994), PECAM 1, EGR 1 (Huang et al., 1997) and KIF 5B (Kamal et al., 2001; Niclas et al., 1994). Within these 7 transcripts, 4 of them went in opposite directions (FKHR, Integrin-5, Oct-3 and PECAM). These inconsistencies in array confirmation have been reported by others (Rajeevan et al., 2001; Tseng et al., 2001; Wang et al., 2001) yet underplayed in many studies. The use of a newer statistical algorithm proposed by the manufacturer (MAS 5.0) did not change this situation. Interestingly, when PCA was used on the array data and compared with PCA on the same targets processed with scqmRT-PCR (FIG. 5B), the 2 populations were separated as expected. However, within the PCA analysis performed on array data, the 2 intermediary cases failed to be separated from the controls (FIG. 5C). The data again highlighted the importance of validation steps following functional genomic approaches.

### 2. Experimental Procedures

### a) Human Brain Tissues

Postmortem human brain tissues from superior frontal gyrus were obtained from the brain bank at University of Rochester. All cases were characterized based on clinical and neuropathological criteria as presented in Table 1.

TABLE 1
Gender, age, post-mortem delay and clinical dementia
rate of the cases used in this study.

| | | | | |
|---|---|---|---|---|
| | Age | Gender | PMD* | |
| Cases | (Yr) | (M/F) | (Hr) | CDR** |

Control

| | | | | |
|---|---|---|---|---|
| C1 (99A-112) | 57 | M | 7.5 | 0 |
| C2 (97A-179) | 66 | M | 5.33 | 0 |
| C3 (97A-191) | 91 | M | 6.1 | 0 |
| C4 (97A-212) | 73 | F | 10.2 | 0 |
| C5 (97A-237) | 87 | F | 10 | 0.5*** |

AD

| | | | | |
|---|---|---|---|---|
| A1 (97A-224) | 86 | M | 5 | 5 |
| A2 (98A-030) | 77 | F | 8.55 | 3 |
| A3 (98A-077) | 84 | F | 5.35 | 5 |
| A4 (98A-175) | 84 | F | 9.1 | 3 |
| A5 (99A-017) | 87 | F | 5 | 3 |

*PMD, Post Mortem Delay,
**Clinical Dementia Rate,
***Retrospective CDR.

### b) RNA Extraction

Total RNA from 200 mg human brain tissue homogenates was extracted using RNeasy Protect Midi Kit (Qiagen). Each RNA preparation also included DNase I and proteinase K (Qiagen) treatment according to the manufacturer's instructions. Yield of total RNA was determined by absorbance at 260 nM. RNA integrity was assessed by both 260/280 nM ratios (ranging from 1.98 to 2.02) and agarose gel electrophoresis.

### c) Reverse Transcription

1 μg total RNA from each sample was reverse transcribed into cDNA in a final volume of 20 μl containing 4 units Omniscript reverse transcriptase (Qiagen) in the manufacturer's buffer, 0.5 mM of each dNTP, 10 units RNase inhibitor (Promega), and 1 μM NVd(T)'s (5′TTTTTTTTTTTTTTTTTTTVN3′). The reactions took place at 37° C. for 12 hours and then stored at −20° C. until further use.

### d) Single Channel Multiplex Quantitative PCR

Real-time PCR reactions were performed using Amplifluor Universal Detection system (Intergen) and iCycler (BioRad). PCR primers were designed using Primer3 software (available at http://www-genome.wi.mit.edu/genome_software/other/primer3.html) to specifically amplify between 177 and 237 base pairs for the genes of interest in the same PCR conditions and were synthesized by Invitrogen. For each gene of interest, an additional forward primer was ordered which contained a “Z-sequence” (ACTGAACCTGACCGTACA), or any other sequence that functions like a Z sequence, at the 5′ end required for UniPrimer annealing. Sequences of the PCR primers are shown in FIG. 7 and FIG. 8. The Amplifluor Universal Detection system kit is based on sunrise primer strategy. The UniPrimer contains the same “Z-sequence”, labeled with a reporter (FAM=6-carboxy-fluorescein) at 5′ and a quencher dye (DABSYL=4-(dimethylamine)azo benzene sulfonic acid) at 3′ of Z-sequence.

For the first round of multiplex quantitative PCR, each 100 μl PCR reaction contained 1 μl cDNA or plasmid, 5 units HotStarTaq DNA polymerase (Qiagen) in the manufacturer's buffer, 0.5 mM of each dNTP, 2 μl of primer mixture. The primer mixture was made of forward and reverse primers for all the genes of interest, at a final concentration of 10 μM each. The forward primers used here did not contain the Z-sequence. The PCR program consisted of 15 minutes at 95° C. to activate the polymerase, followed by 15 cycles of 20 seconds of denaturation at 95° C., 20 seconds of annealing at 60° C. and 35 seconds of elongation at 72° C. A final step of elongation at 72° C. for 10 min was performed. This round of PCR was pre-amplification only and did not involve real-time PCR.

For the second round of multiplex quantitative PCR, each 50 μl real-time PCR reaction contained 1 μl of first round multiplex quantitative PCR reaction, 2.5 units HotStarTaq DNA polymerase (Qiagen) in the manufacturer's buffer, 0.5 mM of each dNTP, 0.02 μM forward primer and 0.2 μM reverse primer for one gene, and 0.2 μM UniPrimer. The PCR program consisted of 15 minutes at 95° C. to activate the polymerase, followed by 50 cycles of 20 seconds of denaturation at 95° C., 20 seconds of annealing at 60° C. and 35 seconds of elongation at 72° C. A final step of elongation at 72° C. for 10 min was performed. Fluorescence intensity was measured during the annealing step of each cycle, so that unincorporated UniPrimers were predominantly in the quenched hairpin conformation. Threshold cycle (C_{T}) for each reaction was analyzed using iCycler software (BioRad).

All real-time PCR experiments were carried out in triplicates and the average C_{T} for the triplicates was used in all subsequent analysis. Reactions omitting enzyme or template were used as negative controls. All reactions were resolved in 1% agarose gel to confirm the PCR specificity. The amount of transcripts was calculated by reference to respective standard curves.

### e) Regular Quantitative PCR

Reaction mixture and conditions were the same as the second round of multiplex quantitative PCR, except that the PCR template was 1111 of plasmid.

### f) Cloning and Constructing Standard Curves

Regular PCR was performed using cDNA as template and the same set of primers for each gene of interest. The forward primers used did not contain Z-sequence. Each PCR product was cloned into pGEM-T Easy vector (Promega). Plasmids were quantified by absorbance at 260 nM. Eight 10-fold serial dilutions of plasmids for each gene of interest were used as templates to perform multiplex quantitative PCR individually in triplicates. Thus a standard curve was constructed for each gene of interest. A linear relationship between the threshold cycles and the log value of input plasmid DNA copy number was observed over the range of 10′ to 10⁸ copies.

### g) Microarray

Double-stranded DNA was synthesized from 15 μg total RNA by using one primer containing poly (dT) and the other primer containing T7 polymerase promoter sequence. In vitro transcription with the double-stranded DNA as a template in the presence of biotinylated UTP and CTP was carried out using the protocol provided by Affymetrix. Biotinylated cRNA was purified, fragmented, and hybridized to HuGeneFL arrays following manufacturer's manual. The hybridized arrays were then washed and stained with streptavidin-phycoerythrin, and scanned with a Hewlett Packard Gene Array Scanner. Data analysis was performed using Affymetrix Genechip Expression Analysis software (version 3.1 and 5.0). Internal controls of housekeeping genes and a test chip were run prior to test samples.

### h) Principle Component Analysis

Data from multiplex quantitative PCR and microarray were first transformed into Excel files, and then imported into S-Plus statistical software package (Insight) as data files. Principle component analysis was performed with either all or selected variables using default settings in S-Plus. The first two principle components were used to make the scatter plots. A screenplot and a loading bar graph were also generated in each analysis by the software.

1. A method of determining the relative copy number of a group of target nucleic acid molecules present in a sample, comprising 1) performing a first PCR in a PCR mixture, wherein the mixture comprises a group of target nucleic acid molecules and a set of first PCR primer pairs, wherein each primer pair is designed to amplify a region of one of the target nucleic acid molecules in the group of target nucleic acid molecules, wherein the first PCR produces a first set of target nucleic acid products, 2) performing a second PCR in a PCR mixture, wherein the mixture comprises an aliquot of the first group of PCR products and a second PCR primer pair which is designed to amplify one of the target nucleic acid products, wherein the second PCR produces a second target nucleic acid PCR product related to one of the target nucleic acid molecules, and 3) quantifying the number of copies of the second target nucleic acid product present in the sample containing the target nucleic acid molecule.

2. A method for quantifying the copy number of a group of target nucleic acids in a sample comprising 1) performing a first PCR in a first PCR mixture, wherein the PCR is performed with at least two different primer pairs, wherein each primer pair is specific for a different target gene expression transcript, 2) performing a second PCR for each target nucleic acid, wherein the second PCR comprises a second primer pair, wherein the second PCR comprises an aliquot of the first PCR mixture, 3) comparing the amount of starting material of each second PCR for each target nucleic acid.

3. A method of quantifying the copy number of a group of target nucleic acids in a sample, comprising 1) performing a first PCR comprising a first set of PCR primer pairs that produces a set of first PCR products, 2) performing a second PCR comprising a second primer pair specific for one of the target nucleic acids and an aliquat of the first set of PCR products that produces a second PCR product, 3) comparing the copy number of each target nucleic acid present in the second PCR.

4. A method of quantifying the copy number of a group of target nucleic acids in a sample, comprising 1) performing a first PCR comprising a first set of PCR primer pairs that produces a set of first PCR products, 2) performing a second PCR comprising a second primer pair and an aliquat of the first set of PCR products that produces a second PCR product, 3) comparing the threshold cycle of the second PCR to a standard curve, wherein the standard curve plots threshold cycle versus copy number of DNA.

5. The method of claim 4, wherein each first PCR primer pair comprises one forward primer and one reverse primer.

6. The method of claim 5, wherein the forward and reverse primers are about equimolar.

7. The method of claim 4, wherein each first PCR primer pair is about equimolar to each of the other first PCR primer pairs in the set of first PCR primer pairs.

8. The method of claim 4, wherein each first PCR primer has about a 50% GC content.

9. The method of claim 4, wherein the first PCR is started by a hot-start.

10. The method of claim 4, wherein the first PCR is performed for less than or equal to 15 cycles.

11. The method of claim 4, wherein the products produced from the target nucleic acid molecules are between 100 and 1000 nucleotides long.

12. The method of claim 4, wherein the products produced from the target nucleic acid molecules are between 20 and 1500 nucleotides long.

13. The method of claim 4, wherein the products produced from the target nucleic acid molecules are between 177 and 237 nucleotides long.

14. The method of claim 4, wherein a first target nucleic acid product is less than 250 nucleotides long.

15. The method of claim 4, wherein each first target nucleic acid product is less than 250 nucleotides long.

16. The method of claim 4, wherein the first PCR is performed with at least 19 PCR primer pairs.

17. The method of claim 4, wherein the second PCR is performed with one primer pair.

18. The method of claim 4, wherein the primer pair in step 2 is different than the any of the primer pairs in step 1.

19. The method of claim 4, wherein the second PCR primer pair is a primer pair present in the set of first PCR primer pairs.

20. The method of claim 4, wherein the second PCR primer pair amplifies the same region of the target nucleic acid as the first PCR primer pair.

21. The method of claim 4, wherein the primer pairs in step 2 comprise a universal primer sequence.

22. The method of claim 4, wherein the set of first PCR products was derived from at least 5 different target nucleic acid molecules.

23. The method of claim 4, wherein the set of first PCR products was derived from at least 15 different target nucleic acid molecules.

24. The method of claim 4, wherein one of the primers from the second PCR primer pair comprises a sequence involved with fluorescent detection.

25. The method of claim 24, wherein the fluorescent detection comprises use of a fluorescent reporter probe selected from the group consisting of SYBR green, Taqman probe, Molecular Beacon, Scorpion Primer, Sunrise Primer, and Eclispe Probe.

26. The method of claim 24, wherein the fluorescence reporter probe is coupled with a quencher.

27. The method of claim 4, further comprising producing cDNA related to the target nucleic acid molecules before performing the first PCR.

28. The method of claim 27, further comprising producing RNA prior to producing the cDNA.

29. A method of determining whether a subject is at risk of acquiring Alzheimer's disease comprising performing the method of claim 1 on a sample from the subject and comparing the copy number of the group of target nucleic acids in the subject to the group of target nucleic acids in a control.

30. The method of claim 29, wherein the first set of primer pairs comprises a pair specific for at least one gene selected from the group consisting of AP180, PP2CB, Dynamin, Syntaxin, PARG, CAMKG, and ICAM5.

31. The method of claim 29, wherein the first set of primer pairs comprises a pair specific for at least two genes selected from the group consisting of AP180, PP2CB, Dynamin, Syntaxin, PARG, CAMKG, and ICAM5.

32. The method of claim 29, wherein the first set of primer pairs comprises a pair specific for at least three genes selected from the group consisting of AP180, PP2CB, Dynamin, Syntaxin, PARG, CAMKG, and ICAM5.

33. The method of claim 29, wherein the first set of primer pairs comprises a pair specific for at least four genes selected from the group consisting of AP180, PP2CB, Dynamin, Syntaxin, PARG, CAMKG, and ICAM5.

34. The method of claim 29, wherein the first set of primer pairs comprises a pair specific for at least five genes selected from the group consisting of AP180, PP2CB, Dynamin, Syntaxin, PARG, CAMKG, and ICAM5.

35. The method of claim 29, wherein the first set of primer pairs comprises a pair specific for at least six genes selected from the group consisting of AP180, PP2CB, Dynamin, Syntaxin, PARG, CAMKG, and ICAM5.

36. The method of claim 29, wherein the first set of primer pairs comprises a pair specific for AP180, PP2CB, Dynamin, Syntaxin, PARG, CAMKG, and ICAM5.

37. The method of claim 29, wherein the first set of primer pairs comprises a pair selected from the group consisting of pair 1, pair 2, pair 3, pair 4, pair 5, pair 6, and pair 7, and wherein pair 1 is SEQ ID NO:17 and 36, wherein pair 2 is SEQ ID NO:13 and 32, wherein pair 3 is SEQ ID NO:19 and 38, wherein pair 4 is SEQ ID NO:18 and 37, wherein pair 5 is SEQ ID NO: 14 and 33, wherein pair 6 is SEQ ID NO: 10 and 29, and wherein pair 7 is SEQ ID NO:3 and 22.

38. The method of claim 29, wherein the first set of primer pairs comprises two pairs selected from the group consisting of pair 1, pair 2, pair 3, pair 4, pair 5, pair 6, and pair 7, and wherein pair 1 is SEQ ID NO:17 and 36, wherein pair 2 is SEQ ID NO:13 and 32, wherein pair 3 is SEQ ID NO:19 and 38, wherein pair 4 is SEQ ID NO:18 and 37, wherein pair 5 is SEQ ID NO: 14 and 33, wherein pair 6 is SEQ ID NO:10 and 29, and wherein pair 7 is SEQ ID NO:3 and 22.

39. The method of claim 29, wherein the first set of primer pairs comprises three pairs selected from the group consisting of pair 1, pair 2, pair 3, pair 4, pair 5, pair 6, and pair 7, and wherein pair 1 is SEQ ID NO: 17 and 36, wherein pair 2 is SEQ ID NO: 13 and 32, wherein pair 3 is SEQ ID NO:19 and 38, wherein pair 4 is SEQ ID NO:18 and 37, wherein pair 5 is SEQ ID NO:14 and 33, wherein pair 6 is SEQ ID NO:10 and 29, and wherein pair 7 is SEQ ID NO:3 and 22.

40. The method of claim 29, wherein the first set of primer pairs comprises four pairs selected from the group consisting of pair 1, pair 2, pair 3, pair 4, pair 5, pair 6, and pair 7, and wherein pair 1 is SEQ ID NO:17 and 36, wherein pair 2 is SEQ ID NO:13 and 32, wherein pair 3 is SEQ ID NO: 19 and 38, wherein pair 4 is SEQ ID NO:18 and 37, wherein pair 5 is SEQ ID NO: 14 and 33, wherein pair 6 is SEQ ID NO: 10 and 29, and wherein pair 7 is SEQ ID NO:3 and 22.

41. The method of claim 29, wherein the first set of primer pairs comprises five pairs selected from the group consisting of pair 1, pair 2, pair 3, pair 4, pair 5, pair 6, and pair 7, and wherein pair 1 is SEQ ID NO:17 and 36, wherein pair 2 is SEQ ID NO:13 and 32, wherein pair 3 is SEQ ID NO:19 and 38, wherein pair 4 is SEQ ID NO:18 and 37, wherein pair 5 is SEQ ID NO:14 and 33, wherein pair 6 is SEQ ID NO:10 and 29, and wherein pair 7 is SEQ ID NO:3 and 22.

42. The method of claim 29, wherein the first set of primer pairs comprises six pairs selected from the group consisting of pair 1, pair 2, pair 3, pair 4, pair 5, pair 6, and pair 7, and wherein pair 1 is SEQ ID NO: 17 and 36, wherein pair 2 is SEQ ID NO: 13 and 32, wherein pair 3 is SEQ ID NO:19 and 38, wherein pair 4 is SEQ ID NO:18 and 37, wherein pair 5 is SEQ ID NO: 14 and 33, wherein pair 6 is SEQ ID NO: 10 and 29, and wherein pair 7 is SEQ ID NO:3 and 22.

43. The method of claim 29, wherein the first set of primer pairs comprises pair 1, pair 2, pair 3, pair 4, pair 5, pair 6, and pair 7, and wherein pair 1 is SEQ ID NO: 17 and 36, wherein pair 2 is SEQ ID NO:13 and 32, wherein pair 3 is SEQ ID NO:19 and 38, wherein pair 4 is SEQ ID NO: 18 and 37, wherein pair 5 is SEQ ID NO: 14 and 33, wherein pair 6 is SEQ ID NO: 10 and 29, and wherein pair 7 is SEQ ID NO:3 and 22.


SEQUENCE LISTING (109 sequences)

SEQ ID NO: 1 - DNA, 20 bp, Artificial Sequence
  Description of Artificial Sequence/note = synthetic construct
actgggacgacatggagaaa

SEQ ID NO: 2 - DNA, 20 bp, Artificial Sequence
  Description of Artificial Sequence/note = synthetic construct
gccatgtaagtcccatcagg

SEQ ID NO: 3 - DNA, 20 bp, Artificial Sequence
  Description of Artificial Sequence/note = synthetic construct
gctcgacagacatcagcaca

SEQ ID NO: 4 - DNA, 20 bp, Artificial Sequence
  Description of Artificial Sequence/note = synthetic construct
ctggagagccatttcctcaa

SEQ ID NO: 5 - DNA, 20 bp, Artificial Sequence
  Description of Artificial Sequence/note = synthetic construct
cagacctacacccgctacca

SEQ ID NO: 6 - DNA, 20 bp, Artificial Sequence
  Description of Artificial Sequence/note = synthetic construct
taccgctacacctgggactt

SEQ ID NO: 7 - DNA, 20 bp, Artificial Sequence
  Description of Artificial Sequence/note = synthetic construct
ctccagccaacttcaccatc

SEQ ID NO: 8 - DNA, 20 bp, Artificial Sequence
  Description of Artificial Sequence/note = synthetic construct
tgcgacatctgtggaagaaa

SEQ ID NO: 9 - DNA, 20 bp, Artificial Sequence
  Description of Artificial Sequence/note = synthetic construct
atggagccaatttctcgtgt

SEQ ID NO: 10 - DNA, 20 bp, Artificial Sequence
  Description of Artificial Sequence/note = synthetic construct
gtgccattgaggtcttcaca

SEQ ID NO: 11 - DNA, 20 bp, Artificial Sequence
  Description of Artificial Sequence/note = synthetic construct
gccaatggagccaagtgtat

SEQ ID NO: 12 - DNA, 20 bp, Artificial Sequence
  Description of Artificial Sequence/note = synthetic construct
cttactccgtgctgtgtcca

SEQ ID NO: 13 - DNA, 20 bp, Artificial Sequence
  Description of Artificial Sequence/note = synthetic construct
ccacagggatgttgcctagt

SEQ ID NO: 14 - DNA, 20 bp, Artificial Sequence
  Description of Artificial Sequence/note = synthetic construct
agctgagcgagatgtggttt

SEQ ID NO: 15 - DNA, 20 bp, Artificial Sequence
  Description of Artificial Sequence/note = synthetic construct
agacctgccttggtgtctgt

SEQ ID NO: 16 - DNA, 20 bp, Artificial Sequence
  Description of Artificial Sequence/note = synthetic construct
agacctgccttggtgtctgt

SEQ ID NO: 17 - DNA, 20 bp, Artificial Sequence
  Description of Artificial Sequence/note = synthetic construct
gagatgcctttgcagcttct

SEQ ID NO: 18 - DNA, 20 bp, Artificial Sequence
  Description of Artificial Sequence/note = synthetic construct
tttgagcaggtggaggagat

SEQ ID NO: 19 - DNA, 20 bp, Artificial Sequence
  Description of Artificial Sequence/note = synthetic construct
ccaaggagaactgcctcatc

SEQ ID NO: 20 - DNA, 20 bp, Artificial Sequence
  Description of Artificial Sequence/note = synthetic construct
cagaggcgtacagggatagc

SEQ ID NO: 21 - DNA, 22 bp, Artificial Sequence
  Description of Artificial Sequence/note = synthetic construct
aaagaaagacaccaagccattt

SEQ ID NO: 22 - DNA, 20 bp, Artificial Sequence
  Description of Artificial Sequence/note = synthetic construct
gtcaggtttcccagagtgga

SEQ ID NO: 23 - DNA, 21 bp, Artificial Sequence
  Description of Artificial Sequence/note = synthetic construct
tgtggtggagtgtccctactc

SEQ ID NO: 24 - DNA, 20 bp, Artificial Sequence
  Description of Artificial Sequence/note = synthetic construct
ctctgcttcagccctgtctt

SEQ ID NO: 25 - DNA, 20 bp, Artificial Sequence
  Description of Artificial Sequence/note = synthetic construct
gagccattgaccttgatgct

SEQ ID NO: 26 - DNA, 20 bp, Artificial Sequence
  Description of Artificial Sequence/note = synthetic construct
atgacctcaaactgggcatc

SEQ ID NO: 27 - DNA, 19 bp, Artificial Sequence
  Description of Artificial Sequence/note = synthetic construct
gggatgggtatgaggtggt

SEQ ID NO: 28 - DNA, 20 bp, Artificial Sequence
  Description of Artificial Sequence/note = synthetic construct
gaggtagaccctggcctctg

SEQ ID NO: 29 - DNA, 20 bp, Artificial Sequence
  Description of Artificial Sequence/note = synthetic construct
aataagctgtgccagggtgt

SEQ ID NO: 30 - DNA, 20 bp, Artificial Sequence
  Description of Artificial Sequence/note = synthetic construct
catatccggcttggttagga

SEQ ID NO: 31 - DNA, 20 bp, Artificial Sequence
  Description of Artificial Sequence/note = synthetic construct
gccttatccaacgcactcat

SEQ ID NO: 32 - DNA, 20 bp, Artificial Sequence
  Description of Artificial Sequence/note = synthetic construct
tgcggtgttcagagaattga

SEQ ID NO: 33 - DNA, 20 bp, Artificial Sequence
  Description of Artificial Sequence/note = synthetic construct
caggactcgacagcatggta

SEQ ID NO: 34 - DNA, 20 bp, Artificial Sequence
  Description of Artificial Sequence/note = synthetic construct
cctcgttgttcccattcact

SEQ ID NO: 35 - DNA, 20 bp, Artificial Sequence
  Description of Artificial Sequence/note = synthetic construct
acctcgttgttcccattcac

SEQ ID NO: 36 - DNA, 20 bp, Artificial Sequence
  Description of Artificial Sequence/note = synthetic construct
ctggtctcaggtggcttcat

SEQ ID NO: 37 - DNA, 20 bp, Artificial Sequence
  Description of Artificial Sequence/note = synthetic construct
atggactgctcgatgctctt

SEQ ID NO: 38 - DNA, 20 bp, Artificial Sequence
  Description of Artificial Sequence/note = synthetic construct
gcttgttctccagcacatca

SEQ ID NO: 39 - DNA, 38 bp, Artificial Sequence
  Description of Artificial Sequence/note = synthetic construct
actgaacctgaccgtacaactgggacgacatggagaaa

SEQ ID NO: 40 - DNA, 38 bp, Artificial Sequence
  Description of Artificial Sequence/note = synthetic construct
actgaacctgaccgtacagccatgtaagtcccatcagg

SEQ ID NO: 41 - DNA, 38 bp, Artificial Sequence
  Description of Artificial Sequence/note = synthetic construct
actgaacctgaccgtacagctcgacagacatcagcaca

SEQ ID NO: 42 - DNA, 38 bp, Artificial Sequence
  Description of Artificial Sequence/note = synthetic construct
actgaacctgaccgtacactggagagccatttcctcaa

SEQ ID NO: 43 - DNA, 38 bp, Artificial Sequence
  Description of Artificial Sequence/note = synthetic construct
actgaacctgaccgtacacagacctacacccgctacca

SEQ ID NO: 44 - DNA, 38 bp, Artificial Sequence
  Description of Artificial Sequence/note = synthetic construct
actgaacctgaccgtacataccgctacacctgggactt

SEQ ID NO: 45 - DNA, 38 bp, Artificial Sequence
  Description of Artificial Sequence/note = synthetic construct
actgaacctgaccgtacactccagccaacttcaccatc

SEQ ID NO: 46 - DNA, 38 bp, Artificial Sequence
  Description of Artificial Sequence/note = synthetic construct
actgaacctgaccgtacatgcgacatctgtggaagaaa

SEQ ID NO: 47 - DNA, 38 bp, Artificial Sequence
  Description of Artificial Sequence/note = synthetic construct
actgaacctgaccgtacaatggagccaatttctcgtgt

SEQ ID NO: 48 - DNA, 38 bp, Artificial Sequence
  Description of Artificial Sequence/note = synthetic construct
actgaacctgaccgtacagtgccattgaggtcttcaca

SEQ ID NO: 49 - DNA, 38 bp, Artificial Sequence
  Description of Artificial Sequence/note = synthetic construct
actgaacctgaccgtacagccaatggagccaagtgtat

SEQ ID NO: 50 - DNA, 38 bp, Artificial Sequence
  Description of Artificial Sequence/note = synthetic construct
actgaacctgaccgtacacttactccgtgctgtgtcca

SEQ ID NO: 51 - DNA, 38 bp, Artificial Sequence
  Description of Artificial Sequence/note = synthetic construct
actgaacctgaccgtacaccacagggatgttgcctagt

SEQ ID NO: 52 - DNA, 38 bp, Artificial Sequence
  Description of Artificial Sequence/note = synthetic construct
actgaacctgaccgtacaagctgagcgagatgtggttt

SEQ ID NO: 53 - DNA, 38 bp, Artificial Sequence
  Description of Artificial Sequence/note = synthetic construct
actgaacctgaccgtacaagacctgccttggtgtctgt

SEQ ID NO: 54 - DNA, 38 bp, Artificial Sequence; identical to SEQ ID NO: 53
  Description of Artificial Sequence/note = synthetic construct

SEQ ID NO: 55 - DNA, 38 bp, Artificial Sequence
  Description of Artificial Sequence/note = synthetic construct
actgaacctgaccgtacagagatgcctttgcagcttct

SEQ ID NO: 56 - DNA, 38 bp, Artificial Sequence
  Description of Artificial Sequence/note = synthetic construct
actgaacctgaccgtacatttgagcaggtggaggagat

SEQ ID NO: 57 - DNA, 38 bp, Artificial Sequence
  Description of Artificial Sequence/note = synthetic construct
actgaacctgaccgtacaccaaggagaactgcctcatc

SEQ ID NO: 58 - DNA, 21 bp, Artificial Sequence
  Description of Artificial Sequence/note = synthetic construct
agatgctgacccatacctcaa

SEQ ID NO: 59 - DNA, 20 bp, Artificial Sequence
  Description of Artificial Sequence/note = synthetic construct
tgaactacctggaccgcttc

SEQ ID NO: 60 - DNA, 20 bp, Artificial Sequence
  Description of Artificial Sequence/note = synthetic construct
cagattgcagagctgttgga

SEQ ID NO: 61 - DNA, 20 bp, Artificial Sequence
  Description of Artificial Sequence/note = synthetic construct
gcgcacagaggaagagaatc

SEQ ID NO: 62 - DNA, 20 bp, Artificial Sequence
  Description of Artificial Sequence/note = synthetic construct
ccgtggatctctggagtgtt

SEQ ID NO: 63 - DNA, 20 bp, Artificial Sequence
  Description of Artificial Sequence/note = synthetic construct
gccagaaacatcctggtagc

SEQ ID NO: 64 - DNA, 20 bp, Artificial Sequence
  Description of Artificial Sequence/note = synthetic construct
gtggtcattgatggggagac

SEQ ID NO: 65 - DNA, 20 bp, Artificial Sequence
  Description of Artificial Sequence/note = synthetic construct
ttcacccctgaagagtccat

SEQ ID NO: 66 - DNA, 20 bp, Artificial Sequence
  Description of Artificial Sequence/note = synthetic construct
ccgaggagaatgtcaagagg

SEQ ID NO: 67 - DNA, 20 bp, Artificial Sequence
  Description of Artificial Sequence/note = synthetic construct
aagcgccatctcttgaggta

SEQ ID NO: 68 - DNA, 20 bp, Artificial Sequence
  Description of Artificial Sequence/note = synthetic construct
cccagaacaagaaggtgagc

SEQ ID NO: 69 - DNA, 20 bp, Artificial Sequence
  Description of Artificial Sequence/note = synthetic construct
atgtgtgtggagagcgtcaa

SEQ ID NO: 70 - DNA, 20 bp, Artificial Sequence
  Description of Artificial Sequence/note = synthetic construct
ataagccctgtcctccaggt

SEQ ID NO: 71 - DNA, 20 bp, Artificial Sequence
  Description of Artificial Sequence/note = synthetic construct
aacctcctctctgccatcaa

SEQ ID NO: 72 - DNA, 20 bp, Artificial Sequence
  Description of Artificial Sequence/note = synthetic construct
ctgttcaggccccatatgat

SEQ ID NO: 73 - DNA, 20 bp, Artificial Sequence
  Description of Artificial Sequence/note = synthetic construct
tctcttctacctggcgctgt

SEQ ID NO: 74 - DNA, 20 bp, Artificial Sequence
  Description of Artificial Sequence/note = synthetic construct
acctcatgctggacaaggac

SEQ ID NO: 75 - DNA, 20 bp, Artificial Sequence
  Description of Artificial Sequence/note = synthetic construct
gggtttcagttgggaaacaa

SEQ ID NO: 76 - DNA, 20 bp, Artificial Sequence
  Description of Artificial Sequence/note = synthetic construct
ctacctccaccatgccaagt

SEQ ID NO: 77 - DNA, 20 bp, Artificial Sequence
  Description of Artificial Sequence/note = synthetic construct
gacctgccacattcaggagt

SEQ ID NO: 78 - DNA, 20 bp, Artificial Sequence
  Description of Artificial Sequence/note = synthetic construct
agatcctgagctccctgaca

SEQ ID NO: 79 - DNA, 20 bp, Artificial Sequence
  Description of Artificial Sequence/note = synthetic construct
ttccagacacgctatcatgc

SEQ ID NO: 80 - DNA, 20 bp, Artificial Sequence
  Description of Artificial Sequence/note = synthetic construct
gaagatcctcaaccccagtg

SEQ ID NO: 81 - DNA, 20 bp, Artificial Sequence
  Description of Artificial Sequence/note = synthetic construct
tccctggagaagagctacga

SEQ ID NO: 82 - DNA, 20 bp, Artificial Sequence
  Description of Artificial Sequence/note = synthetic construct
atccccaacaacgtgaagac

SEQ ID NO: 83 - DNA, 20 bp, Artificial Sequence
  Description of Artificial Sequence/note = synthetic construct
gaaggtgaaggtcggagtca

SEQ ID NO: 84 - DNA, 20 bp, Artificial Sequence
  Description of Artificial Sequence/note = synthetic construct
actgttgtgcatgctgtggt

SEQ ID NO: 85 - DNA, 20 bp, Artificial Sequence
  Description of Artificial Sequence/note = synthetic construct
agcttgttcaccaggagcag

SEQ ID NO: 86 - DNA, 20 bp, Artificial Sequence
  Description of Artificial Sequence/note = synthetic construct
tccccgtctcccttataacc

SEQ ID NO: 87 - DNA, 20 bp, Artificial Sequence
  Description of Artificial Sequence/note = synthetic construct
caaggcctcattcagctctc

SEQ ID NO: 88 - DNA, 20 bp, Artificial Sequence
  Description of Artificial Sequence/note = synthetic construct
ctcaattggttgggcagatt

SEQ ID NO: 89 - DNA, 20 bp, Artificial Sequence
  Description of Artificial Sequence/note = synthetic construct
cacagcaggacaccaaaaga

SEQ ID NO: 90 - DNA, 20 bp, Artificial Sequence
  Description of Artificial Sequence/note = synthetic construct
acgtcatccgagtccttcac

SEQ ID NO: 91 - DNA, 20 bp, Artificial Sequence
  Description of Artificial Sequence/note = synthetic construct
ggtttaggagggttgcttcc

SEQ ID NO: 92 - DNA, 20 bp, Artificial Sequence
  Description of Artificial Sequence/note = synthetic construct
agcttttgctcctctgcttg

SEQ ID NO: 93 - DNA, 20 bp, Artificial Sequence
  Description of Artificial Sequence/note = synthetic construct
gcgagaaacgtgaacctagc

SEQ ID NO: 94 - DNA, 20 bp, Artificial Sequence
  Description of Artificial Sequence/note = synthetic construct
aattcagaagcctgcaagga

SEQ ID NO: 95 - DNA, 21 bp, Artificial Sequence
  Description of Artificial Sequence/note = synthetic construct
ttcagagacagccaggagaaa

SEQ ID NO: 96 - DNA, 20 bp, Artificial Sequence
  Description of Artificial Sequence/note = synthetic construct
gttgctggtgagtgtgcatt

SEQ ID NO: 97 - DNA, 20 bp, Artificial Sequence
  Description of Artificial Sequence/note = synthetic construct
ccaaagtagacctgcccaga

SEQ ID NO: 98 - DNA, 20 bp, Artificial Sequence
  Description of Artificial Sequence/note = synthetic construct
ttcgacaactttgctgcttg

SEQ ID NO: 99 - DNA, 20 bp, Artificial Sequence
  Description of Artificial Sequence/note = synthetic construct
aagcagcactcatccacgat

SEQ ID NO: 100 - DNA, 20 bp, Artificial Sequence
  Description of Artificial Sequence/note = synthetic construct
accgcacatcatctcgtaca

SEQ ID NO: 101 - DNA, 20 bp, Artificial Sequence
  Description of Artificial Sequence/note = synthetic construct
cacccaagagtcccaaacat

SEQ ID NO: 102 - DNA, 20 bp, Artificial Sequence
  Description of Artificial Sequence/note = synthetic construct
cacacaggatggcttgaaga

SEQ ID NO: 103 - DNA, 20 bp, Artificial Sequence
  Description of Artificial Sequence/note = synthetic construct
gtctttcttgcaggctttgg

SEQ ID NO: 104 - DNA, 20 bp, Artificial Sequence
  Description of Artificial Sequence/note = synthetic construct
cggaggattatcgttggtgt

SEQ ID NO: 105 - DNA, 20 bp, Artificial Sequence
  Description of Artificial Sequence/note = synthetic construct
aatccactggtgaaccaagc

SEQ ID NO: 106 - DNA, 20 bp, Artificial Sequence
  Description of Artificial Sequence/note = synthetic construct
gttgacagcccagcttcttc

SEQ ID NO: 107 - DNA, 20 bp, Artificial Sequence
  Description of Artificial Sequence/note = synthetic construct
agcactgtgttggcgtacag

SEQ ID NO: 108 - DNA, 20 bp, Artificial Sequence
  Description of Artificial Sequence/note = synthetic construct
ctcggtgaactccatctcgt

SEQ ID NO: 109 - DNA, 20 bp, Artificial Sequence
  Description of Artificial Sequence/note = synthetic construct
gacaagcttcccgttctcag